(19) United States
(12) Patent Application Publication
(10) Pub. No.: US 2025/0064083 A1
(43) Pub. Date: Feb. 27, 2025

(54) Modeling Plant Ingredient Proteins for Use in Developing Food Products

(71) Applicant: Frito-Lay North America, Inc., Plano, TX (US)

(72) Inventors: Jenna Wang, Shelton, CT (US); Yi Zhu, Plano, TX (US); Luis Antonio Garay Almada, The Colony, TX (US); Jingting Hui, Purchase, NY (US)

(21) Appl. No.: 18/813,328

(22) Filed: Aug. 23, 2024

Related U.S. Application Data
(60) Provisional application No. 63/534,695, filed on Aug. 25, 2023.

(51) Int. Cl. A23J 3/14; A23L 11/00; G16B 15/20; G16B 40/20

(52) U.S. Cl. CPC A23J 3/14; A23L 11/00; G16B 15/20; G16B 40/20

(57) ABSTRACT

Disclosed are systems and methods for determining a concentration of a plant ingredient in a developed food product before or while making the food product. A method may include: receiving, by a computing system, protein data for the plant ingredient, retrieving at least one model trained to generate output indicating a desired concentration of the plant ingredient to be used in developing the food product, the model including at least one of a pH, temperature, and protein-content model, providing the received protein data as input to the model, receiving the output from the model, and generating instructions for developing the food product including the desired concentration range of the plant ingredient.

### CROSS-REFERENCE TO RELATED APPLICATION

This application claims priority to U.S. Provisional Application No. Ser. No. 63/534,695, filed on Aug. 25, 2023, the entire contents of which is incorporated herein by reference.

### TECHNICAL FIELD

This document generally describes devices, systems, techniques, and methods related to computer-based modeling using protein structures of plant ingredients that contain proteins, such as chickpeas, so that the plant ingredients may be added to formulas or mixtures used for developing food products to achieve higher nutritional value for such developed food products.

### BACKGROUND

Food products may be created and processed using various unit operations and ingredients under differing processing conditions. The food products may include, but are not limited to, snacks, chips, crackers, breads, etc. Some food products may be created and/or processed with ingredients that impart minimal to no impact on taste, such as certain starches and other bulking agents, which typically may not add value from a health/nutrition perspective for consumers.

There is a continuing desire to improve food products from a nutritional/health perspective by incorporating natural or plant-based ingredients (also referred to as plant ingredients) such as legumes, grains, pulses, cereals, beans, nuts, and other plant-basted ingredients with high protein content (e.g., above 20 wt. % wet basis). However, there are many difficulties that must be overcome in order to successfully incorporate plant-based ingredients. Among other things, many plants contain proteins, which, when incorporated with other ingredients and processed may affect the processing of such when seeking to prepare a food product with desirable organoleptic properties. In this regard, the proteins present in plants may include, but may not be limited to metabolic proteins, storage proteins, structural proteins, and/or membrane proteins. Storage proteins may include albumin and globulin, as well as other types of proteins such as prolamins and glutelins. The globulins may further include legumins and/or vicillins.

Protein structural and functional properties can change in various degrees depending on the type and amount of processing during food production. The proteins may change through inter- and/or intra-molecular interactions, including but not limited to covalent bond formation (e.g., disulfide bridging), hydrogen bridging (which may occur between amide, carboxylic, sulfhydryl, and/or hydroxyl groups present in amino acid residue side chains), electrostatic interactions (which may occur between side chain groups that can be ionized in a matrix, such as carboxylate and charged amino groups), hydrophobic interactions, and/or Van der Waals interactions. These interactions may occur during protein denaturation, which in turn modifies properties of the protein surface, as well as exposure/enclosure of internal hydrophobic pockets. These structural changes in protein architecture can have a substantial impact in the processability and functionality of a finished product, as well as the finished product's organoleptic/nutritional properties. For example, changes in formation of intra-molecular disulfide bond bridging can affect functional properties such as protein solubility, protein water holding capacity, protein gelation, protein emulsification, protein foaming, etc. Understanding the effects that processing plant ingredients for use in food product development may be challenging. Sometimes, if processing conditions of the plant ingredients are not appropriately determined, the processed plant ingredients may possess negative organoleptic properties such as consistency, texture, mouthfeel, and/or other consumer-related experiences plant ingredients.

### SUMMARY

Systems, methods, algorithms, techniques, and technology for modeling proteins present in plant ingredients under differing processing conditions to then be used in developing food products are described, achieving higher nutritional value for the food products, and obtaining a mechanistic understanding of plant ingredient protein functionality. Linking the mechanistic behaviors of protein interactions and the effects in product functionality and nutritional profile can be improved with the disclosed modeling techniques to provide added value towards developing and providing better products for consumers. The described modeling techniques can be used to predict and guide protein functionality changes (e.g., solubility, water binding capacity, gelation, etc.) due to inter- and/or intra-molecular bonding and denaturation of the protein, which may impact processability of the protein and developed food products' organoleptic properties. The disclosed modeling techniques may additionally or alternatively be used for formulation and product innovation, as described below.

For example, the disclosed technology may be used to determine how processing conditions such as temperature, pH, protein content, and/or salt content affect proteins present in the plant ingredients. Such determinations may then be used, such as automatically by a computing system, to determine changes in the processing of food ingredients, such as doughs, to which the plant ingredients are added. The disclosed technology may be used to increase nutrition of the developed food products by developing the food products with plant ingredients rich in nutrients, such as protein. For example, the disclosed technology may be used to determine how proteins present in the plant ingredients interact with other ingredients when mixed with and, in some cases, formed into a dough, that may be used to develop food products. Specific dough rheological properties, such as dough thickness, hardness, adhesiveness, cohesiveness, and gumminess are important in the development of food products such as chips, crisps, and dough-based snacks, and may be impacted by the amount of plant ingredients that are rich in protein and are included in the dough. The disclosed technology may be used to determine how much starch to replace with the protein-containing plant ingredients while taking into consideration how the protein present in each plant ingredient has characteristics that may impact overall solubility of the dough. Determinations about how the proteins present in the plant ingredients interact with the other ingredients may be used to further analyze and model solubility of the proteins to improve a formula and other processing conditions used for developing the food products. The disclosed technology may also be used to determine how various processing conditions and parameters, such as pH, protein composition, and/or temperature impact the solubility (as an example of one of functional properties described above) of the proteins and thus the development of the food products, formulation, and food product innovation.

The disclosed technology may be applied to known or contemplated plant ingredients currently used and possibly contemplated for use in making food products. Such plant ingredients may include, but are not limited to, legumes, such as chickpeas, lentils, and peas, as well as cereals, such as corn and/or oats, which may be used in formulas for developing, processing, or otherwise producing different food products, such as chips, breads, crackers, and other types of snacks. It will be appreciated that the plant ingredients may include constituents other than protein such as starches and fibers that may be affected by different processing conditions. These constituents and/or types of processing conditions may be modeled using the disclosed technology. Thus, the skilled artisan will appreciate that the disclosure's reference to proteins likewise includes starch, fiber, fat, and carbohydrates.

It will also be appreciated that the plant ingredients may have any suitable form such as a liquid or solid. When provided as solid, it may be present in any suitable form such a flour or particles, and may be provided in an unprocessed, e.g., raw, or processed state.

The disclosed technology may provide for molecular dynamics simulations and other modeling techniques to improve the processing of food products such as doughs used to form food products by using predicted solubility, or other processing conditions. For example, pH, salt content, temperature, and/or protein content may be modeled for different plant ingredients to determine the effects of those properties on such plant ingredients, that may then be used to improve processing as well as organoleptic/nutritional properties of the developed food products.

As described throughout this disclosure, plant ingredients can be used in processes for developing food products. The plant ingredients may contain plant proteins that may add health/nutritional value to the developed food products. Plant proteins may be meaningful sources of protein (e.g., may contain from 1%, 2%, 3%, 4%, 5%, 6%, 7%, 8%, 9%, 10%, 11%, 12%, 13%, 14%, 15%, 16%, 17%, 18%, 19%, 20 wt % or more protein content on wet basis) which are derived from plants.

One or more of the described embodiments may include a method for determining a concentration of a plant ingredient in a developed food product before or while making the food product, the method may include: receiving, by a computing system, protein data for the plant ingredient; retrieving, by the computing system and from a data store, at least one model that has been trained to generate output indicating a desired concentration of the plant ingredient to be used in developing the food product, the at least one model including at least one of a pH model, a temperature model, and a protein-content model; providing, by the computing system, the received protein data as input to the at least one model; receiving, by the computing system, the output generated by the at least one model, the output indicating the desired concentration range of the plant ingredient to be used in developing the food product; generating, by the computing system, instructions for developing the food product, the instructions including the desired concentration range of the plant ingredient and at least one of (i) an amount of salt based on the desired concentration range of the plant ingredient and (ii) a temperature above or below a threshold temperature value at which to develop the food product with the desired concentration range of the plant ingredient; and returning, by the computing system, the instructions.

In some implementations, the described embodiments may optionally include one or more of the following features. For example, providing, by the computing system, the received protein data to the at least one model may include providing at least a portion of the received protein data to each of the pH model, the temperature model, and the protein-content model. The method may also include: comparing, by the computing system, output from each of the pH model, the temperature model, and the protein-content model, and generating, by the computing system and based on the comparison, an updated desired concentration range of the plant ingredient.

Sometimes, the pH model may be trained and/or was previously trained, by the computing system, to correlate a measured pH level during one or more unit operations performed to produce the developed food product with predetermined concentrations of salt that either reduce or increase solubility of the plant ingredient to determine desired concentrations of different plant ingredients to be included in a formulation to produce different food products. The at least one model may be a deep learning model that may be and/or was trained, by the computing system, to determine a desired concentration for each of a group of different plant ingredients to be used in developing the food product. The group of different plant ingredients may include one or more proteins (and, as noted above, may include any one of, a combination of some or all, or all constituents of plants useful in making food products, e.g. starch, fiber, fats, carbohydrates, etc.). The one or more proteins may be selected from the group consisting of albumin, globulin, prolamin, glutelin, and mixtures thereof.

The method may also include: receiving, by the computing system, at least one processing condition for which the food product is developed and a desired amount of protein present in the developed food product, and providing, by the computing system, the received at least one processing condition and the desired amount of protein present as input to the at least one model. The at least one processing condition can include a temperature in one or more unit operations used for developing the food product. The at least one processing condition may include a pressure level in one or more unit operations used for developing the food product.

As another example, the pH model may be and/or was trained by the computing system to: (i) predict different pH levels to be used when producing the developed food product based on using different concentrations of the plant ingredient, (ii) determine a desired concentration range of the plant ingredient to be used in developing the food product based on the predicted different PH levels of the ingredients used to form a dough used for developing the food product, and (iii) generate output indicating the determined desired concentration range of the plant ingredient. Sometimes, the temperature model may be and/or was trained by the computing system to: (i) correlate (a) temperatures at which the food product is developed with different concentrations of the plant ingredient with (b) solubility of the protein in the plant ingredient to generate a temperature correlation, (ii) determine a desired concentration range of the plant ingredient to be used in developing the food product based on the temperature correlation, and (iii) generate output indicating the determined desired concentration range of the plant ingredient. As another example, the protein-content model may be and/or was trained by the computing system to: (i) correlate (a) proteins and amounts present in each plant ingredient in developing the food product with (b) solubility of the proteins to generate a protein-content correlation, (ii) determine a desired concentration range of the plant ingredient to be used in developing the food product based on the protein-content correlation, and (iii) generate output indicating the determined desired concentration range of the plant.

Sometimes, returning, by the computing system, the instructions may include transmitting the instructions to a user device that may be configured to present at least the desired concentration range of the plant ingredient in a graphical user interface (GUI) display of the user device. Providing, by the computing system, the received protein data to the at least one model may include: selecting one of the pH model, the temperature model, and the protein-content model based on one or more selection criteria, and providing the received protein data to the selected model. The method may also include iteratively training, by the computing system, the at least one model using (i) different concentrations of the plant ingredient and (ii) data resulting from developing the food product according to the instructions. The protein data for the plant ingredient may include at least one of a type of protein associated with the plant ingredient, a structure for the at least one type of protein associated with the plant ingredient, an amino acid sequence for the at least one type of protein associated with the plant ingredient, and physical characteristics of the at least one type of protein associated with the plant ingredient. To this end, the protein data for the plant ingredient may include the types, structure amino acid sequence, and physical characteristics for all the proteins associated with the plant ingredient.

One or more described embodiments may include a method for determining a concentration of a specific plant ingredient, exemplified in this disclosure with reference to chickpeas, in a developed food product before or while making the food product, the method including: receiving, by a computing system, protein data for chickpeas; retrieving, by the computing system and from a data store, at least one model that has been trained to generate output indicating a desired concentration of the chickpeas to be used in developing the food product; providing, by the computing system, the received protein data as input to the at least one model; receiving, by the computing system, the output generated by the at least one model, the output indicating the desired concentration range of the chickpeas to be used in developing the food product, generating, by the computing system, instructions for developing the food product, the instructions including the desired concentration range of the chickpeas, and returning, by the computing system, the instructions.

The method may optionally include one or more of the following features. The at least one model may include at least one of a pH model, a temperature model, and a protein-content model. The at least one model may be and/or was trained using a deep neural network (DNN) to determine desired concentrations of different plant ingredients used for developing different food products, the different plant ingredients including at least the chickpeas. The instructions may include an amount of salt based on the desired concentration range of the chickpeas. The instructions may include a temperature above or below a threshold temperature value at which to develop the food product with the desired concentration range of the chickpea.

One or more described embodiments may include a method for determining a concentration of chickpeas in a developed food product before or while making the food product, the method including: receiving protein data for the chickpeas; retrieving, from a data store, at least one model that has been trained to generate output indicating a desired concentration of the chickpeas to be used in developing the food product, wherein the at least one model includes at least one of a pH model, a temperature model, and a protein-content model; providing the received protein data as input to the at least one model; receiving the output generated by the at least one model, wherein the output indicates the desired concentration range of the chickpeas to be used in developing the food product; generating instructions for developing the food product, wherein the instructions include the desired concentration range of the chickpeas; and returning the instructions.

The method may optionally include one or more of the following features. Providing the received protein data to the at least one model may include providing at least a portion of the received protein data to each of the pH model, the temperature model, and the protein-content model. The method may also include: comparing output from each of the pH model, the temperature model, and the protein-content model and generating, based on the comparison, an updated desired concentration range of the chickpeas. The instructions further may include at least one of (i) an amount of salt based on the desired concentration range of the chickpeas and (ii) a temperature above or below a threshold temperature value at which to develop the food product with the desired concentration range of the chickpeas. The pH model may be trained to correlate a measured pH level during one or more unit operations performed to produce the developed food product with predetermined concentrations of salt that either reduce or increase solubility of the protein in the chickpeas to determine desired concentrations of the chickpeas to be included in a formulation to produce different food products. The at least one model may be a deep learning model that might have been trained to determine a desired concentration for a plurality of different plant ingredients to be used in developing the food product, the plurality of different plant ingredients including the chickpeas. The plurality of different plant ingredients may include one or more proteins selected from the group consisting of albumin, globulin, prolamin, glutelin, and mixtures thereof.

The method may also include receiving at least one processing condition for which the food product is developed and a desired amount of protein present in the developed food product, and providing the received at least one processing condition and the desired amount of protein present as input to the at least one model. The at least one processing condition may include a temperature in one or more unit operations used for developing the food product. The at least one processing condition may include a pressure level in one or more unit operations used for developing the food product.

The pH model might have been trained to: (i) predict different PH levels to be used when producing the developed food product based on using different concentrations of the chickpeas, (ii) determine a desired concentration range of the chickpeas to be used in developing the food product based on the predicted different pH levels of the ingredients used to form a dough used for developing the food product, and (iii) generate output indicating the determined desired concentration range of the chickpeas. The temperature model might have been trained to: (i) correlate (a) temperatures at which the food product is developed with different concentrations of the chickpeas with (b) solubility of the protein in the chickpeas to generate a temperature correlation, (ii) determine a desired concentration range of the chickpeas to be used in developing the food product based on the temperature correlation, and (iii) generate output indicating the determined desired concentration range of the chickpeas. The protein-content model might have been trained to: (i) correlate (a) proteins and amounts present in the chickpeas in developing the food product with (b) solubility of the proteins to generate a protein-content correlation, (ii) determine a desired concentration range of the chickpeas to be used in developing the food product based on the protein-content correlation, and (iii) generate output indicating the determined desired concentration range of the chickpeas. Returning the instructions may include transmitting the instructions to a user device that may be configured to present at least the desired concentration range of the chickpeas in a graphical user interface (GUI) display of the user device. The method may also include iteratively training the at least one model using (i) different concentrations of the chickpeas and (ii) data resulting from developing the food product according to the instructions.

One or more described embodiments may include a method for determining a concentration of a plant ingredient in a developed food product before or while making the food product, the method including: receiving protein data for the plant ingredient; retrieving, from a data store, at least one model that has been trained to generate output indicating a desired concentration of the plant ingredient to be used in developing the food product; providing the received protein data as input to the at least one model; receiving the output generated by the at least one model, wherein the output indicates the desired concentration range of the plant ingredient to be used in developing the food product; generating instructions for developing the food product, wherein the instructions include the desired concentration range of the plant ingredient; and returning the instructions.

The method may optionally include one or more of the following features. For example, the at least one model may include at least one of a pH model, a temperature model, and a protein-content model. The at least one model might have been trained using a deep neural network (DNN) to determine desired concentrations of different plant ingredients used for developing different food products, the different plant ingredients including at least chickpeas. The instructions may include an amount of salt based on the desired concentration range of the plant ingredient. The instructions may include a temperature above or below a threshold temperature value at which to develop the food product with the desired concentration range of the plant ingredient.

The described devices, system, and techniques may provide one or more of the following advantages. For example, the disclosed technology may provide for computationally-efficiently synthesizing and analyzing many data points about different plant ingredients to determine preferred or recommended concentrations of those plant ingredients for use in developing food products. The resulting food products may have increased quantities of nutrients while still maintaining desired consistency, texture, and/or taste. For example, the disclosed technology may be used to determine a desired concentration of the plant ingredient to add in a formula for developing a food product such as a snack chip to increase nutrient level, e.g., the protein content of such plant-based food product. Moreover, the disclosed technology may provide techniques for sustainable formulation of virtually designed formulas for developing the food products, like snack chips.

Traditional systems and methods did not have clarity about particular plant ingredient properties and how those properties (e.g., structural properties, surface properties) would react in different conditions (e.g., processing conditions such as temperature pH, or protein concentration; production conditions). Moreover, traditional systems and methods did not have automated techniques to process large amounts of disparate data about the plant ingredients and their properties, and rather relied upon human testing, trial, and error of only a finite quantity and type of plant ingredient properties. The disclosed techniques, on the other hand, provide for machine-automated and comprehensive distillation, analysis, and correlation of large amounts of data points that may represent any quantity and/or type of plant ingredient properties to accurately and efficiently generate optimal solutions for food product formulation and development. The described models are uniquely trained to perform data processing operations with high accuracy that the human mind is incapable of doing. For example, the human mind may not reasonably receive and process many different data points to accurately and quickly glean insight into optimal solutions for food product formulation and development without requiring additional testing and trial and error, where the gleaned insight may include tactical information such as molecular, geometric, and chemical insight for particular plant ingredient(s) to develop products through a shortened production path. Moreover, the disclosed techniques reduce overall processing power and improve efficiency while using available compute resources. The disclosed techniques shortens a product development path by reducing or otherwise eliminating time and costs as compared to the traditional approaches of testing and trial and error.

In that regard, traditional approaches of testing and trial and error require developing compositional formulas with prior products possibly used as a guide and with formulator experience. Thereafter, the proposed composition may be processed in a bench-scale arrangement to determine the effects of possible processing conditions on the ingredients forming the proposed formula. Based on the results, the proposed composition may be modified with the modified proposed composition being retested. This iterative process continues until a satisfactory product is obtained. Thereafter, the proposed composition may be tested in a pilot plant process using apparatus and processing conditions that mimic those of a commercial processing plant. Based on the results of the pilot plant testing, the composition and/or processing conditions may be modified and the process may be retested. As before, this iterative process continues until satisfactory product and processing conditions are obtained. The skilled artisan will appreciate that the above described process is time consuming and labor intensive.

The disclosed techniques may be used to provide and improve formulation of developed food products as further described. More particularly, the disclosed techniques can be leveraged to perform virtual formulation for developing various food products. Virtual formulation may include molecular modeling-computer-aided formulation design, which may allow thousands of data points to be analyzed and assessed to determine and/or test different formulations, without having to perform actual real-world experiments and formulations. Such techniques may provide faster, efficient, and accurate results and analysis to formulate various different food products.

Similarly, the disclosed modeling techniques may provide mechanisms for understanding impacts of different concentrations of plant ingredients in processing and developing different food products. Accordingly, the disclosed techniques may improve the food product processing and development processes to provide greater or higher quantity of nutrient-filled products to consumers that also achieve desired texture, quality, consistency, and/or taste attributes or otherwise improve consumer acceptability (e.g., texture, mouthfeel). The disclosed technology may provide robust techniques for determining thermal parameter impacts on handling and dough machining capabilities in developing the food products. As a result, improvements may be made to the formulas and the unit operations used to produce the food products that may take into consideration thermal parameter impacts that are modeled or otherwise determined using the disclosed technology.

The thermal parameters may implicate temperature and other physical parameter sensors (e.g., pressure, work imparted to dough, line speeds, roller gap width in sheeting applications, etc.). Sensor data may be tracked, stored, and/or analyzed to help train the described models and algorithms to predict optimal parameters for processing different plant ingredients for purposes of developing food products. Even if a particular plant ingredient, such as chickpeas, may not be dependent on (i.e., affected by) the processing parameters, such parameters and condition readings made by sensors may be correlated to optimal outcomes. The disclosed technology may similarly be used for sensor-related artificial intelligence (AI) and/or analytics.

Protein modeling may also help to reduce development time and resource consumption to create tasty snacks with improved nutritional value (e.g. higher protein content). Through the understanding of protein interactions in-silico, the number of trials and attempts needed during the product development stage may be reduced. The disclosed technology may also reduce time, resource consumption, and impacts of product development testing.

A same model or set of models may be used and applied across different plant ingredients. Model parameters may be adjusted based on the type of plant ingredient (e.g., protein, starch, fiber, fat, carbohydrate, etc.) being modeled. As a result, model building, training, improvements, storage, and/or use may utilize fewer computational resources and processing power, making the disclosed technology leaner against running models for each type of plant ingredient. The same model or set of models may advantageously allow for faster, more comprehensive, and more accurate decisions to be made. Additionally, the same model or set of models may be iteratively trained and improved using a robust set of data under different processing conditions and parameters for the different plant ingredients. Model(s) accuracy may be improved to generate formulas and other processing conditions for incorporating plant ingredients into the food products without compromising quality characteristics of the food products and, in some instances, improving quality characteristics of food products.

Moreover, the disclosed models may be used to predict protein-rich ingredient functionality. The disclosed technology may similarly be used for computer dynamic modeling to design protein containing plant ingredients with desired functionality. For example, the claimed and disclosed technology may also be used to suggest certain processing parameters, e.g. temperature, pressure, time, concentration of the plant ingredient or other ingredient amounts to ensure a greater or optimal protein amount. Furthermore, the disclosed technology may also be used to improve accuracy of predicting dough thermodynamic properties, where traditionally, temperature impact on protein processing may be challenging to simulate.

As another example, the disclosed technology may leverage graph neural networks and multi-dimensional data to generate robust and accurate model outcomes/results. Graph neural networks can provide linkage between various data points in various dimensions. Using graph neural networks allows for data to be converted into high dimensional space, which can help fill in gaps around sparse data points and provide a more robust data set for use with the disclosed models. Such multi-dimensional data collection, interpolation, and analysis can provide improved modeling outcomes/results in comparison to using two dimensional (2D) traditional data, such as tabular data.

For ease of understanding, the described systems, methods, algorithms, techniques, and technology for modeling proteins present in plant ingredients under different processing conditions will be described with reference to chickpeas and chickpea protein. It should be understood that reference to or mention of chickpeas will refer likewise to other plant ingredients, unless specifically noted otherwise or unless it is evident from the context that the reference to chickpea is meant to refer to only chickpeas. In addition, unless the context suggests otherwise, reference to chickpea(s) and chickpea protein should be understood to refer to the solid form of chickpea, which is typically provided as a flour or particles.

In addition, the following description may refer to food product(s) and developed food product(s), and it is intended to refer to both the finished food product, e.g., a snack food product suitable for human consumption, and to the mixture or dough that may be further processed to form a snack food product suitable for human consumption.

All recited percentages refer to a percent by weight, unless otherwise stated or the context makes clear that it does not refer to a percent by weight.

The details of one or more implementations are set forth in the accompanying drawings and the description below. Other features and advantages will be apparent from the description and drawings, and from the claims.

### BRIEF DESCRIPTION OF THE DRAWINGS

FIG. 1 is a conceptual diagram for determining information for developing a food product using plant ingredients.

FIG. 2A is a flowchart of a process for determining information for developing a food product using plant ingredients.

FIG. 2B is a flowchart of a process for determining information for developing a food product using chickpeas.

FIG. 3 is a flowchart of a process for modeling plant ingredient data to develop food products.

FIG. 4 illustrates graphs of pH and salt content impacts for example plant proteins Albumin and Globulin using the disclosed techniques.

FIG. 5 illustrates graphs of temperature impact for example disulfide and hydrophobic distances using the disclosed techniques.

FIG. 6 illustrates graphs of protein content impact for example disulfide and hydrophobic aggregation, interaction, and solubility.

FIG. 7 is a flowchart of a process for training at least one model to predict plant ingredient conditions for developing food products.

FIG. 8 is a schematic diagram that shows an example of a computing device and a mobile computing device.

FIG. 9 is a schematic diagram of exemplary methods of making a snack food according to one embodiment.

FIG. 10 illustrates graphs depicting results from modeling dough mixtures with various ratios of protein:starch.

Like reference symbols in the various drawings indicate like elements.

### DETAILED DESCRIPTION

This document generally relates to technology for modeling different plant ingredients that may be used to develop food products and to determine processing conditions or other parameters for developing the food products with concentrations of the modeled plant ingredients. The disclosed technology may train, iteratively improve, and implement various models that have been trained to determine and/or predict processing conditions for different plant ingredients according to parameters such as, but not limited to, pH, salt content, protein content, and/or temperature.

The disclosed technology may be applied to various plant ingredients, including but not limited to grains, pulses, cereals, beans, nuts, and the like such as chickpeas, oats, and/or corn. Plant-based proteins may respond similarly to temperature parameters, which allows for temperature modeling to be used in determining preferred processing conditions for the ingredients used to create the food products (including plant ingredients). The plant ingredients may be included with other ingredients using the disclosed technology to improve the nutritional value of the developed food products while maintaining consumer-related organoleptic experiences when eating the food products (e.g., mouthfeel, texture, consistency, feel, taste).

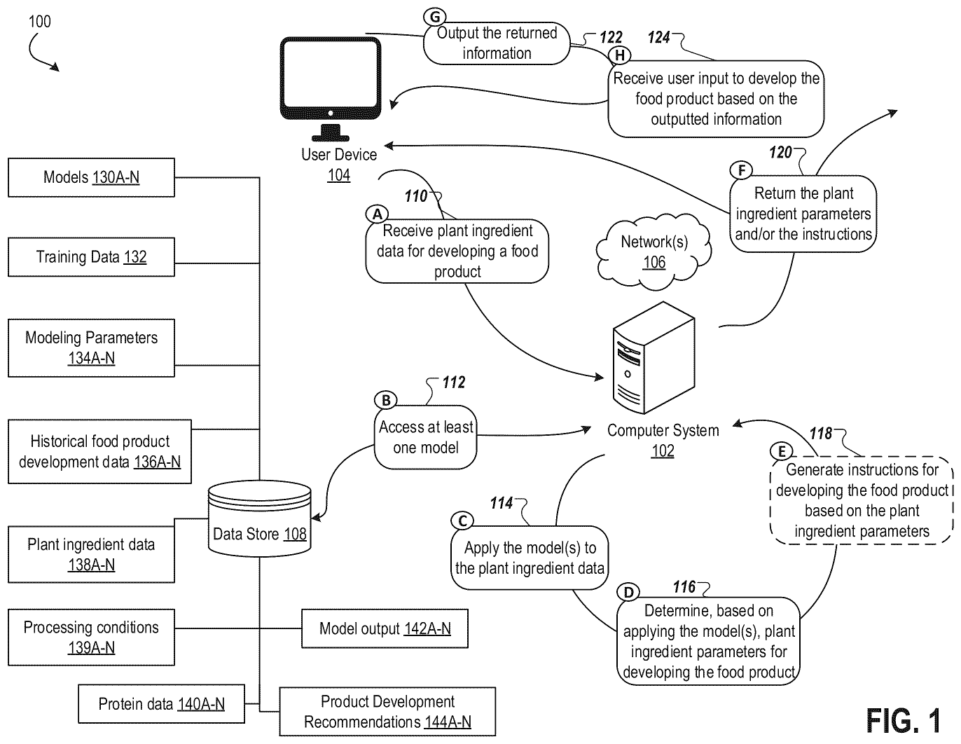

Referring to the figures, FIG. 1 is a conceptual diagram of a system 100 for determining information for developing a food product using plant ingredients. More particularly, the system 100 illustrates using computer models to enhance plant protein-based food product development processes. The disclosed technology may be used for modeling processing conditions of chickpea protein and other protein-containing plant ingredients that may be used to develop different food products, such as snacks, as described herein. The system 100 may include a computer system 102, a user device 104, and/or a data store 108, which may communicate (e.g., wired, wirelessly) via network(s) 106.

The computer system 102 may be any type of computing system, network of computing devices or systems, cloud-based system, and/or computing device. The computer system 102 may be configured to determine processing conditions for developing food products with plant ingredients, formulas for developing the food products, and/or recommendations for improving or adjusting the processing conditions and/or ingredient formulas in the development of the food products. The computer system 102 may be configured to train, iteratively improve, and/or implement one or more machine learning models that may be used to perform the disclosed techniques. The computer system 102 may be configured to determine how various parameters and processing conditions impact processing of plant ingredients in the development of the food products using the machine learning-trained models. In some implementations, the computer system 102 may be part of the user device 104 and/or the data store 108.

The user device 104 may be any type of user computing device, mobile device, mobile phone, smartphone, laptop, tablet, and/or computer. The user device 104 may include input and output devices (e.g., display, touch screen, keyboard, mouse, microphone, speakers, haptic feedback) which may be used to receive input from relevant users and present information to the users. The user device 104 may be used, as an illustrative example, by a user who develops the food products, creates or modifies the formulas and/or processes for developing the food products, and/or studies/determines how and what plant ingredients may be used for developing the food products. As described herein, the user device 104 may present in one or more graphical user interface (GUI) displays information including but not limited to: (i) modeling outputs, (ii) graphical or other illustrative displays of the modeling outputs, (iii) recommendations for improving or modifying the formulas for developing the food products, (iv) recommendations for quantities and/or other parameters/processing conditions of the plant ingredients to be used in developing the food products, etc.

The data store 108 may be any type of data storage device, system, database, cloud-based storage, RAM, ROM, and/or memory. The data store 108 may be configured to store a variety of data, including but not limited to models 130A-N described herein, training data 132, modeling parameters 134A-N, historical food product development data 136A-N, plant ingredient data 138A-N, processing conditions 139A-N, protein data 140A-N, model output 142A-N, product development recommendations 144A-N, etc. The data store 108 may also maintain any other type of data, which may be captured, detected, and/or collected across a network of machines, sensors, devices, computing systems, etc. Sometimes, the data collected across the network may be analyzed in real-time or near real-time, as the data is captured, detected, and/or collected (instead of or in addition to storing the collected data in the data store 108).

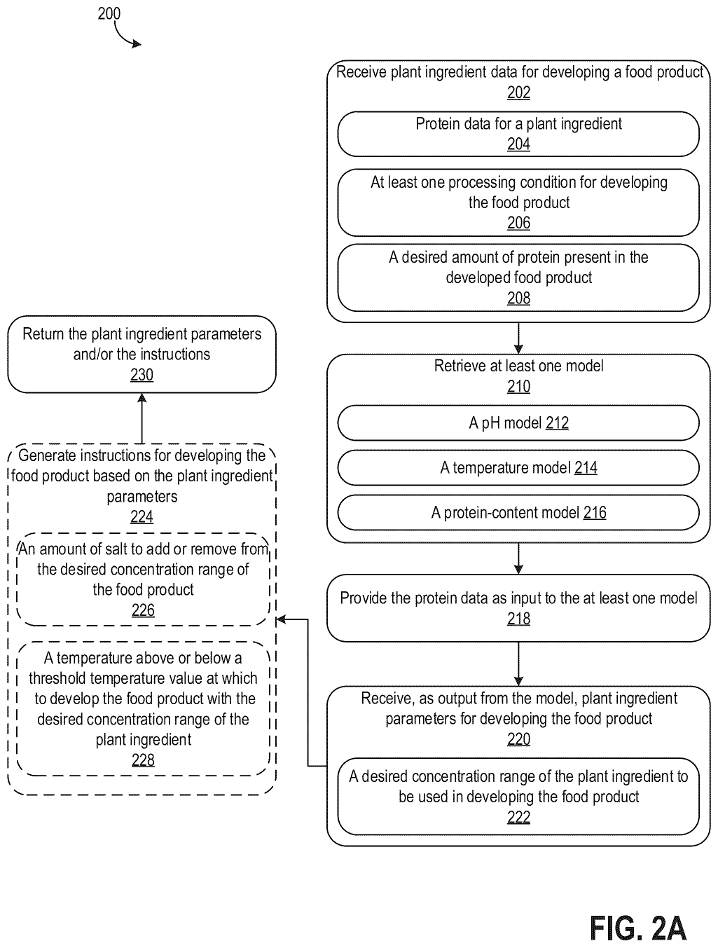

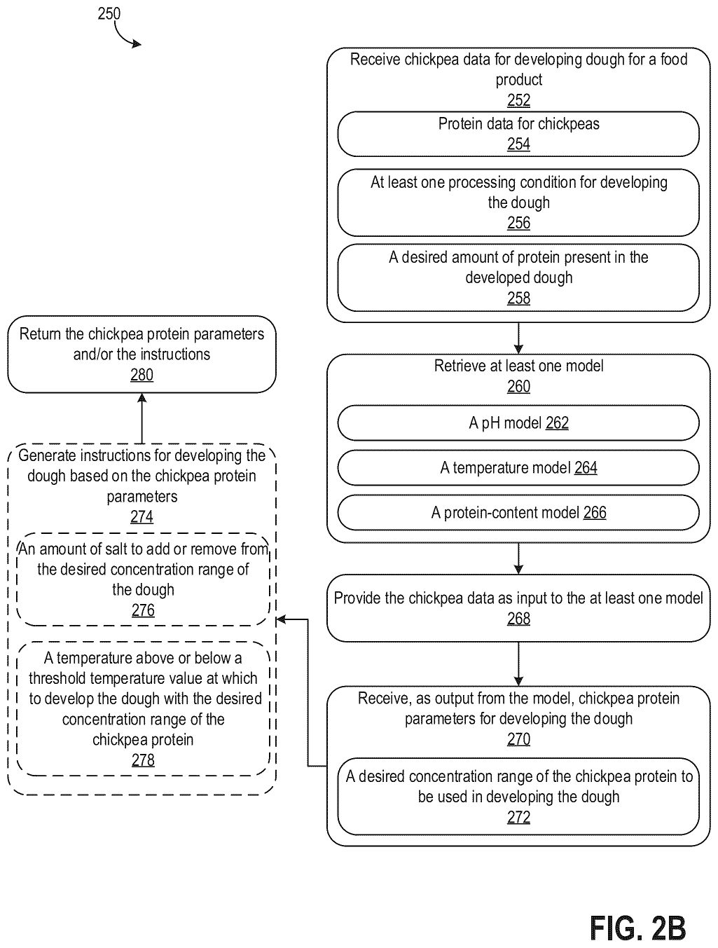

Still referring to FIG. 1, the computer system 102 may receive plant ingredient data 138A-N for developing a food product from a user device 104 (block A, 110). Refer to the process 200 in FIG. 2A for further discussion about the plant ingredient data. In brief, the data may include protein data for the plant ingredient (e.g., type of protein, quantity or concentration of the protein), processing condition(s) for developing the food product, and/or desired amounts of protein sought to be present in the food product once developed (and/or during development).

The computer system 102 may access at least one of the models 130A-N from the data store 108 in block B (112). The models 130A-N may include pH, temperature, and/or protein-content models. The models 130A-N may include one or more deep learning models or other neural networks or machine learning models. Refer to discussion below for further details about the models 130A-N.

In block C (114), the computer system 102 may apply the model(s) to the plant ingredient data 138A-N. The plant ingredient data 138A-N and/or a portion thereof may be provided to the model(s) as input. Sometimes, one or more other data from the data store 108 may be provided as model inputs, including but not limited to the modeling parameters 134A-N, the historical food product development data 136A-N, the processing conditions 139A-N, and/or the protein data 140A-N. Refer to at least the process 200 in FIG. 2A for further discussion about applying the model(s).

The computer system 102 may determine, based on applying the model(s), plant ingredient parameters (e.g., the modeling parameters 134A-N, the processing conditions 139A-N) for developing the food product (block D, 116). The parameters may include recommendations for processing conditions during the food product development. The processing conditions may include processing temperature, processing pressure levels, processing time, concentration of the plant ingredient(s) to add to a formula for developing the food product, and/or other modifications or adjustments to the formula or processing conditions for developing the food product.

Optionally, the computer system 102 may generate instructions (e.g., the product development recommendations 144A-N) for developing the food product based on the determined plant ingredient parameters (block E, 118). The instructions, as described further below, may include recommendations for adjusting a temperature during the food product development. The instructions may include recommendations for adjusting a level or quantity of salt in the formula or other processing conditions for the food product. One or more other instructions may also be generated, as described herein.

The computer system 102 may return the plant ingredient parameters and/or instructions in block F (120). For example, the computer system 102 may store the returned information in the data store 108. The information may then be retrieved at one or more future times, provided to the user device 104, used for iteratively training and/or improving the model(s), and/or used for determining additional instructions and/or recommendations for developing the food product using the plant ingredient.

As another example, the computer system 102 may transmit the returned information to the user device 104. The user device 104 may then output the returned information in one or more GUIs (block G, 122). The user device 104 may output one or more graphs indicating the modeled parameters (e.g., impact of temperature, pH, and/or protein-content on plant ingredient protein solubility or other characteristics). The user device 104 may output one or more recommended actions to be taken by the user in modifying the formula for developing the food product and/or otherwise processing the plant ingredient for use in developing the food product. The user device 104 may also receive user input to develop the food product based at least in part on the outputted information (block H, 124). Sometimes, the user input may be provided to the computer system 102 and used to improve, retrofit, or otherwise iteratively improve the model(s). The user input may be used as additional inputs to one or more of the models to accurately determine processing conditions and/or other information about the plant ingredient and/or the development of the food product.

The disclosed techniques may also be used for other or additional ingredients and may include information such as plant protein ratios in formulations where protein is present with other constituents of either or both of the other ingredients and the plant ingredients, such as starch and fiber. The techniques may also be used for additives such as emulsifiers, texturizers, antioxidants, enzymes and the like. The disclosed techniques may also be used with respect to various processing conditions including, but not limited to, humidity, water uptake, expansion, and/or extrusion (e.g., high pressure). Sensor data that is used by the disclosed techniques may be collected using Internet of Things (IoT) devices, controllers, or other similar edge devices. This sensor data may be collected from one machine, component thereof, and/or any local, regional, and/or global network of machines or components thereof.

In some implementations, the described model(s) may be run on an edge device without having to transfer the sensor data between devices, storage systems, and/or cloud-based systems and/or storage. For example, humidity levels may be measured in real-time using edge sensors, then fed into the model for real-time prediction at the edge. As a result, output can be generated quickly and accurately while using minimal lightweight processing power/resources to develop food products. Moreover, the model(s) may be run on the cloud, to allow for more robust data collection from various different devices. Sometimes, the disclosed techniques may be performed in a hybrid mode, either offline (e.g., performed at a remote computing system) and/or on the cloud/on the edge, which can depend based on network and/or other resource availability. This hybrid mode approach may advantageously avoid network downtime and/or sharing of too much network traffic during production time to ensure the disclosed techniques may continue to be performed (rather than delayed). Then, when the network and/or resource availability returns to one or more predetermined threshold availability conditions/levels, data and/or model determinations/outputs can be uploaded back to the cloud.

FIG. 2A is a flowchart of a process 200 for determining information for developing a food product using plant ingredients. The process 200 may be performed before the plant ingredients are processed. Therefore, the process 200 may be performed to predict processability of the plant ingredients, e.g., to predict the ease or difficulty expected to be encountered if the plant ingredients were actually processed according to the various parameters. Based on the modeling described herein, the plant ingredients can then actually be processed to develop the food product.

As an illustrative example, the process 200 can be performed when performing a design of an experiment (DOE). Variables such as temperature, pH, salt concentration, or other variables included in the disclosed model, can be processed to define specific responses that may link with processing variables. One example is protein solubility, which may correlate with dough machinability. The process 200 may be used to determine an educated DOE to include plant protein (and may include an amount of such plant protein) in the dough, and obtain a desired outcome for developed food product(s). As another example, the process 200 may be performed during a reformulation of an existing product. To change certain product attributes or enhance certain attributes, the disclosed techniques may also be used. In terms of fine-tuning processing conditions, protein modeling as described in the process 200 may be performed to help guide processing changes through mechanistic understanding. Typically, processing changes may result in intermediate or finished products that require analytical tools for characterization. Computer modeling as described herein may reduce an amount of samples and analysis required at a processing crossroad.

The process 200 may be performed by the computer system 102. The process 200 may also be performed by one or more other computing systems, devices, computers, networks, cloud-based systems, and/or cloud-based services. For illustrative purposes, the process 200 is described from the perspective of a computer system.

Referring to the process 200 in FIG. 2A, the computer system may receive plant ingredient data for developing a food product in block 202. At least one model may be trained to generate output indicating a desired concentration of the plant ingredient to be used in developing the food product. The computer system may receive protein data for a plant ingredient (block 204), at least one processing condition for developing the food product (block 206), and/or a desired amount of protein present in the developed food product (block 208). The protein data may include, for example, a type of protein associated with the plant ingredient, a structure of the protein associated with the plant ingredient, a sequence (e.g., amino acid sequence) for the protein associated with the plant ingredient, and/or physical characteristics of the protein associated with the plant ingredient.

In block 210, the computer system may retrieve at least one model from a data store or local memory/storage. The at least one model may include a pH model (block 212), a temperature model (block 214), and/or a protein-content model (block 216).

The pH model (block 212) may be trained, such as by the computer system or another computing system, to correlate pH levels of the mixture (plant ingredient together with other ingredients) or dough used to form the developed food product with predetermined concentrations of salt that may reduce and/or increase solubility of the ingredients or dough used to form the developed food product. The pH model (block 212) may therefore be trained to determine desired concentrations of different plant ingredients to be used in developing different food products. In some implementations, the pH model (block 212) may be trained to (i) predict different pH levels to be used when producing the developed food product based on different concentrations of the plant ingredient, (ii) determine a desired concentration range of the plant ingredient to be used in developing the food product based on the predicted different PH levels of the ingredients or dough used to form the developed food product, and/or (iii) generate output indicating the determined desired concentration range of the plant ingredient. The pH model may be generated using physical modeling techniques.

Sometimes, the pH model may be built or otherwise generating by molecular modeling. This modeling may be achieved with knowledge about different plant ingredient protein structures and compositions. Molecular modeling provides insight into how molecules interact with each other and with their environments, their structure, and their function(s). For example, molecular modeling techniques may indicate protein stability of the plant ingredient. Proteins may change their structure and stability in response to different pH levels. Molecular modeling may help predict how plant ingredient proteins fold and whether they become more or less stable at different pH levels. As another example, molecular modeling may help understand protein charge and conformation. The charge of proteins may be influenced by pH of their environment. Molecular modeling may depict how changes in pH affect ionization of amino acid side chains, which in turn may impact the protein's overall charge, conformation, and interactions with other molecules. For example, acidic or basic conditions can lead to unfolding or aggregation of proteins. As yet another example, solubility of proteins may be pH-dependent. Modeling may predict how pH changes affect the solubility of the plant ingredient proteins, which may be important for their functional properties in food products. For instance, proteins may precipitate or dissolve at specific pH values, thereby influencing texture and clarity. Moreover, functional properties of proteins, such as gelation, emulsification, and foaming, may be influenced by pH. Molecular modeling may provide insights into how pH affects the protein's ability to perform these functions, which may also be useful for optimizing food formulations and processing conditions. Similarly, some plant ingredients (e.g., chickpeas) may contain various enzymes that may be sensitive to pH changes. Molecular modeling may therefore help understand how pH affects enzyme structure and activity, which may be relevant for applications in food processing described herein.

The temperature model (block 214) may be trained to (i) correlate (a) temperatures at which the food product is developed with different concentrations of the plant ingredient with (b) solubility of the protein (one, some, or all of the proteins) in the plant ingredient. The temperature model (block 214) may also be trained to (ii) determine a desired concentration range of the plant ingredient to be used in developing the food product based on the correlations. The temperature model (block 214) may be trained to generate output indicating the determined desired concentration range of the plant ingredient.

Sometimes, the temperature model may be built using molecular dynamics (MD) simulations (e.g., molecular modeling). This approach may provide detailed insights into dynamic behavior of molecular systems, such as how temperature affects structure and properties of the plant ingredient proteins, which in turn may help optimize processing conditions. For example, one or more proteins of the plant ingredient may be identified as relevant for processing conditions. A model of the identified protein(s) may be generated, including the protein's three-dimensional structure(s). Sometimes a solvent (e.g., water) and/or ions (e.g., to mimic physiological conditions) may be added to create a realistic environment for the protein(s). MD simulations may be set up at various temperatures or temperature ranges to determine how the temperature(s) affects the protein(s). Further analysis may be performed using results from the simulations to identify how temperature affects the protein(s): structure(s), stability, flexibility, and/or functional properties. Additional visualization techniques may be used to examine molecular dynamic trajectories and observe conformational changes or aggregation patterns of the protein(s) more directly. Based on the simulation results generated and analyzed by the temperature model, ideal temperature ranges that maintain protein stability and functionality may be identified. Such temperature ranges may be used to guide determinations of optimal processiong conditions for the plant ingredient in food products.

The protein-content model (block 216) may be trained to (i) correlate (a) proteins and amounts present in each plant ingredient product in developing the food product with (b) solubility of the protein (one, some, or all of the proteins) in the plant ingredient. The protein-content model (block 216) may be trained to (ii) determine a desired concentration range of the plant ingredient to be used in developing the food product based on the correlations. The protein-content model (block 216) may also be trained to generate output indicating the determined desired concentration range of the plant ingredient. As described above in reference to the pH model and the temperature model, the protein-content model may similarly be built using MD simulations.

In some implementations, the retrieved model may be a deep learning model that was trained, by the computer system or another computing system, to determine a desired concentration for each of a plurality of different plant ingredients to be used in developing the food product. The plant-based proteins may be selected from a group consisting of albumin, globulin, prolamin, glutelin, and/or mixtures thereof. The deep learning model may be trained to calculate, determine, or otherwise predict a variety of different parameters for each of the plurality of plant ingredients, instead of predicting one parameter at a time or predicting parameters for one plant ingredient at a time. The deep learning model may therefore be used to predict or otherwise determine parameters for all different plant ingredients. The deep learning model may also be trained to receive, as inputs, a variety of different data, such as tabular data and/or graphs. Consequently, multi-dimensional data (e.g., matrices of different pH, temperature, crystal structures, etc.) may be modeled and handled appropriately using the deep learning model. The deep learning model may advantageously provide rich information for more than one protein structure. The deep learning model may leverage a fingerprint of each protein structure to figure out (i.e., elucidate) thousands or more data points about the protein structure. These data points may be provided as input to the model to generate a large and robust data set for use in iteratively improving the model and/or generating more accurate model output. The deep learning model may be trained to predict or otherwise determine plant ingredient levels/ranges.

Deep learning models may be developed with various configurations, including but not limited to dense layers, loss function, and drop-out layers to optimize and product accurate results while minimizing probability of overfitting. Sequential models, for example, are a type of deep learning model and may be used with the disclosed techniques. Example sequential models include Recurrent Neural Networks (RNNs), Long Short-Term Memory (LSTM) networks, or Gated Recurrent Units (GRUs), all of which may be well-suited for time-series, or sequential data. The sequential model may include a stack of single-input and single-output layer(s) to train each model for predicting pH, temperature, and protein-content model. The model may also include hidden layers, such as LSTM or GRU layers, to capture sequential dependencies and relationships in inputted data. A sequential model may also be trained and used to predict all three targets (pH, temperature, and protein-content) together, using three dense layers as the output layer.

An activation function of Rectified Linear Unit (ReLu) may be used, which introduces non-linearity into a deep learning model while being computationally efficient. In a neural network, ReLU may be applied to the output of each neuron (or node) in a layer. This activation function may determine whether the neuron should be activated or not based on its input value. By stacking multiple layers with ReLU activations, neural networks may learn and model complex patterns in data.

A Mean Absolute Error (MAE) loss function may be used for evaluating performance of the model by quantifying how close predicted values are to actual values. A lower MAE, for example, may indicate better performance than a higher MAE. Advantageously, MAE may be less sensitive to outliers compared to other techniques (since it does not square the errors). MAE also may provide a straightforward interpretation of average prediction errors.

An Adaptive Moment Estimation (Adam) optimizer may be used. Adam may be known for its efficiency and effectiveness in handling large datasets and complex models. Adam may adapt learning rates for each parameter based on estimates of first and second moments of the gradients (e.g., mean and uncentered variance). This allows Adam to adjust the learning rate dynamically during training. Adam is also computationally efficient and requires minimal memory overhead compared to other adaptive methods. Adam may also perform well across a variety of architectures and hyperparameters, and it generally converges faster than traditional stochastic gradient descent.

In some implementations, other machine learning models may also be leveraged to predict all three targets (pH, temperature, and protein-content) within a single model, such as XGBoost model.

In some implementations, one or more of the models may be network-based models, such as convolutional neural networks (CNNs). One or more other modeling techniques and/or infrastructures may be used for modeling other plant ingredients. Moreover, model output and/or data retrieved from sensors or physics-based models can be fed into the model(s) (e.g., deep learning model) for data augmentation. Such iterative training may reduce the cost and process for collecting new data and then training the model(s) on the newly collected data.

The computer system may use all retrieved models. For example, the computer system may model all three parameters (pH, temperature, and protein-content) to then determine how to modify all the parameters or any combination thereof. In some implementations, modeling only one of the parameters may not be enough to generate accurate recommendations for developing the food product with the plant ingredient. For example, simply increasing temperature during food product development may not reduce stickiness of the dough used to form the resulting food product to achieve a desired consistency or texture of the food product. As another example, the model can be a deep learning model that can perform multi-variable predictions. In other words, the deep learning model can be trained to predict pH, temperature, protein-content, or any combination thereof, rather than training and deploying multiple models that generate one variable prediction.

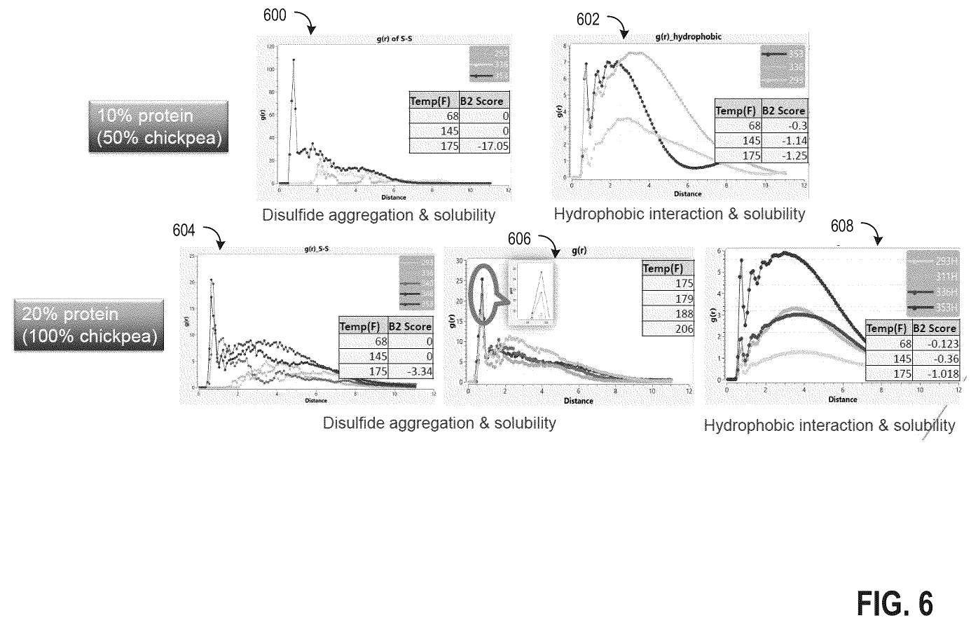

The computer system may leverage the multiple models to optimize different parameters used for developing the food product. As an illustrative example, if a protein level is high (e.g., exceeds some threshold level) and temperature is increased during food product development, then an aggregation level may increase by a small amount (e.g., less than a threshold amount). This means that adding more chickpea protein or other protein-containing plant ingredient, even if the temperature is increased, may not contribute to reducing the stickiness of the dough used to form the resulting food product. Other parameters, such as pH may therefore be modeled and used to adjust a product formula containing the chickpea protein to achieve a desired dough property, e.g., stickiness. In some implementations, other parameters may be considered, such as adding an emulsifier, to reduce surface polar area and achieve the desired property(ies), e.g., stickiness for the dough used to form the developed food product. Refer to graphs 602, 604, and 608 in FIG. 6 for further discussion.

As another illustrative example, both pH and temperature may be modeled separately, and then only one of the resulting outputs may be used/chosen during runtime/real-time development of the food product. Both pH and temperature may not need to be combined in order to determine a desired product formula for developing the food product with the plant ingredient protein(s). Temperature modeling, on the one hand, may use all the same pH values and achieve the same outputs as pH modeling, where the pH modeling may use all different pH values. Sometimes, one or more of the models may be used to verify or validate the output from one or more of the other models. The computer system may use a portion of the models. The computer system may use one of the models. Sometimes, one or more of the models may be used to iteratively train and improve one or more of the other models.

The computer system may provide the protein data (or a portion thereof) as input to at least one model in block 218. For example, the computer system may provide at least a portion of the protein data to each of the pH model, the temperature model, and the protein-content model. The computer system may provide different portions of the protein data to each of the models. Sometimes, the computer system may provide a portion of the protein data to only some of the models. In some implementations, the computer system may select one of the pH models, the temperature models, and the protein-content models based on one or more selection criteria, and then provide the received protein data to the selected model. One or more selection criteria may be based on the plant ingredient and/or the protein present in the plant ingredient. As an illustrative example, certain proteins may be modeled more accurately based on temperature versus pH and/or protein content. The type of protein may therefore be used to select a preferred model. As another example, one or more selection criteria may be based on a type of food product being developed. One or more selection criteria may be based on a type of data available for modeling in the process 200. For example, if the type of data available includes sensor temperature data that is collected during development of the food product using the plant ingredient, then the computer system may select and use the temperature model as the preferred model. One or more other factors and considerations may be used for selecting which model to be used with the disclosed techniques.

Sometimes, the computer system may provide additional or other data to the model(s) as input. The computer system may receive at least one processing condition for which the food product is developed and/or a desired amount of protein present in the developed food product. The computer system may then provide the received at least one processing condition and/or the desired amount of protein present as input to the model(s). Sometimes, at least one processing condition may include cooking conditions of the plant ingredient (e.g., chickpeas), such as temperature and/or time of cooking. At least one processing condition may include a sheeting operation. The sheeting operation may include additional information such as a cooking temperature used to treat the plant protein ingredient, which may dictate a rheology of the resulting dough needed to manufacture the food product. At least one processing condition may include a cooking time that may be required to modulate the resulting dough rheology used to develop the food product.

The computer system may receive, as output from the model, plant ingredient parameters for developing the food product (block 220). For example, the output may include a desired concentration range of the plant ingredient to be used in developing the food product (block 222). Sometimes, the computer system may provide at least a portion of the protein data to each of the retrieved models. The computer system may then compare output from each of the retrieved models and generate, based on the comparison, an updated desired concentration range of the plant ingredient. The updated desired concentration range may be an average, mean, or other aggregated number for the desired concentration ranges determined by each of the retrieved models. In some implementations, the updated desired concentration range may be a desired concentration range that was generated by the retrieved models and has a highest corresponding confidence value. In other words, each of the models may be configured to generate a confidence value indicating a likelihood that the model's output (e.g., the desired concentration range) is accurate or most likely for developing the particular food product. Sometimes, a model generating output having the highest confidence value may be used to train or otherwise iteratively improve the other models (thereby increasing accuracy of the other models in generating corresponding outputs). In some implementations, if one or more of the models achieve at least a desired level of confidence and/or accuracy, that model(s) may be used alone or in combination with other models to generate accurate output.

Optionally, the computer system may generate instructions for developing the food product based on the plant ingredient parameters (block 224). For example, the computer system may optionally determine an amount of salt to be present in the ingredients and/or dough used to form the food product (block 226). As another example, the computer system may optionally determine a temperature above or below a threshold temperature value at which to develop the food product within the desired concentration range of the plant ingredient (block 228).

In block 230, the computer system may return the plant ingredient parameters and/or instructions. Returning the instructions may include transmitting the instructions to a user device that may be configured to present at least the desired concentration range of the plant ingredient in a GUI display of the user device. In some implementations, returning the instructions may include transmitting the model output to the user device for presentation in the GUI display. A user at the user device may then determine, based on the model output, one or more instructions for developing the food product, such as modifications to a formula for developing the food product and/or processing conditions (e.g., temperature, pressure, cooking/processing time, etc.) for developing the food product.

In some implementations, as described further below, the model output(s) can be used for formulating food products with chickpea proteins. Ingredient composition can be complex and the model output(s) for various formulations can be different. For example, various starch components (e.g., amylose, amylopectin), additives, water, oil, and/or enzymes can change dough physicochemical and thermal properties. One or more similar techniques can be used to determine a ratio of proteins to starches or a ratio of proteins to fibers to determine an impact on the dough physicochemical properties.

Moreover, from an AI and/or deep learning model perspective, the model output(s) can be fed into a feedback loop for continuous learning and training. A validation control can be implemented to monitor model shift(s). As a result, if the model starts to not perform well, additional model training, generation of new model(s), and/or human intervention can be identified. Relevant users can also be notified accordingly. These techniques can similarly alert relevant users if and when data drifts from expected results, such as resulting ingredients for food products being different than previous similar ingredients.

FIG. 2B is a flowchart of a process 250 for determining information for developing a food product using chickpeas. The process 250 may be performed by the computer system 102. The process 250 may also be performed by one or more other computing systems, devices, computers, networks, cloud-based systems, and/or cloud-based services. For illustrative purposes, the process 250 is described from the perspective of a computer system.

Referring to the process 250 in FIG. 2B, the computer system may receive chickpea data for developing a food product in block 252. The food product may be, for example, chips, other snacks, and/or dough that may be used for developing the chips or other snacks. For example, the computer system may receive protein data for the chickpeas (block 254), which may include concentrations of proteins such as albumin and/or globulin.

The computer system may receive at least one processing condition for developing the dough, chips, or other snacks (block 256). The processing conditions may include, for example, a temperature and/or pressure applied to ingredients used to create the dough, a salt concentration for developing the products, an amount of time that the temperature and/or pressure is applied to the products to create the dough, a quantity of the chickpeas added to the dough, etc.

The computer system may additionally or alternatively receive a desired amount of protein present in the dough (block 258). The desired amount may indicate an amount of nutritional value and/or nutrients that a user would like to be present in the dough or the resulting chips, snacks, or other food products. The desired amount may indicate an amount of protein from the chickpeas or other plant ingredients such as legumes in various physical forms (e.g., as whole products (e.g., seeds) or in the form of flours) to add to the dough for processing. The desired amount may indicate a resulting amount of protein from the chickpeas or other plant ingredients such as legumes in various physical forms (e.g., as whole products (e.g., seeds) or in the form of flours) to remain in the dough once the processing is complete. Refer to blocks 202-208 in the process 200 of FIG. 2A for further discussion.

In block 260, the computer system may retrieve at least one model from a data store or local memory/storage. At least one model may include a pH model (block 262), a temperature model (block 264), and/or a protein-content model (block 266). Refer to blocks 210-216 in the process 200 of FIG. 2A for further discussion.

The computer system may provide the chickpea data (or a portion thereof) as input to at least one model in block 268. Refer to block 218 in the process 200 of FIG. 2A for further discussion.

The computer system may receive, as output from the model, chickpea protein parameters for developing the dough (block 270). For example, the output may include a desired concentration range of the chickpea protein to be used in developing the dough (block 272). Sometimes, the output may include a concentration range of the chickpea itself to be used in the dough, where using the concentration range of the chickpea when forming the dough may result in the dough having the desired amount of protein. Refer to blocks 220-222 in the process 200 of FIG. 2A for further discussion.

Optionally, the computer system may generate instructions for developing the dough based on the chickpea protein parameters (block 274). For example, the computer system may optionally determine an amount of salt to add or remove from the desired concentration range of the dough (block 276). As another example, the computer system may optionally determine a temperature above or below a threshold temperature value at which to make the dough within the desired concentration range of the chickpea protein (block 278). Refer to blocks 224-228 in the process 200 of FIG. 2A for further discussion.

In block 280, the computer system may return the chickpea protein parameters and/or instructions. Refer to block 230 in the process 200 of FIG. 2A for further discussion.

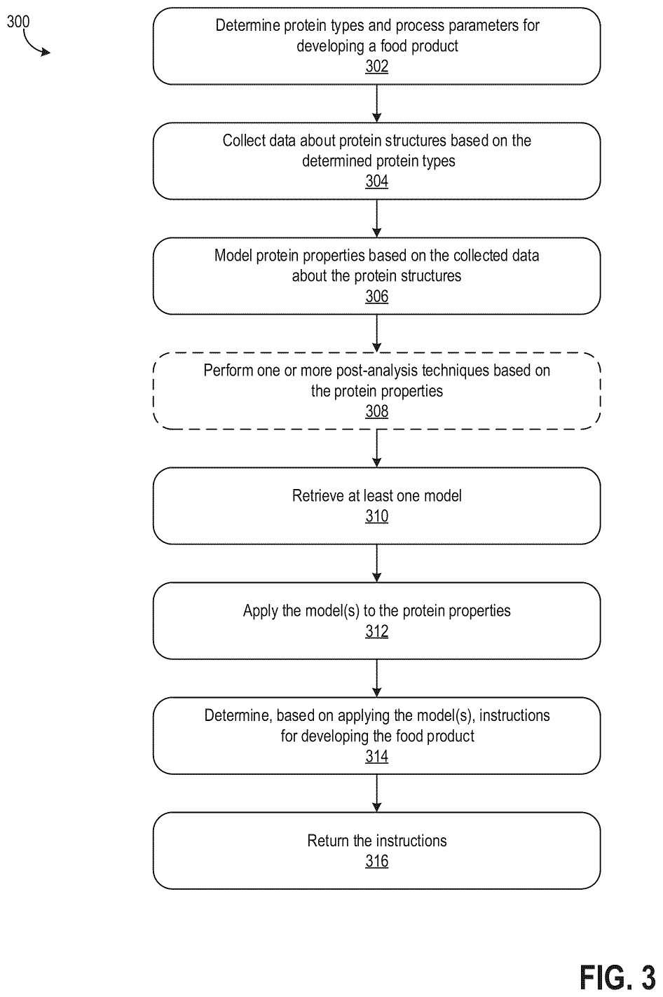

FIG. 3 is a flowchart of a process 300 for modeling plant ingredient data to develop food products. The process 200 may be performed by the computer system 102. The process 300 may also be performed by one or more other computing systems, devices, computers, networks, cloud-based systems, and/or cloud-based services. For illustrative purposes, the process 300 is described from the perspective of a computer system.

Referring to the process 300 in FIG. 3, the computer system may determine protein types and/or process parameters for developing a food product in block 302. The process parameters may include, for example, pH, temperature, salt content, protein content, etc.

The computer system may collect data about protein structures based on the determined protein types in block 304. For example, the computer system may receive data such as x-ray structures of proteins in a particular plant ingredient. When x-ray data is captured, computer vision technology (e.g., image analysis) from an AI perspective can be leveraged, not simply tabular data-related modeling techniques. The x-ray data may include compounded information, not simply tabular data. Using the x-ray data, which can include images and/or graphs, additional information can be embedded therein, and extracted using computer vision technology and/or AI techniques to draw more analysis from the data. The x-ray data, such as images, can be segmented. Semantic techniques may also be applied to the x-ray data to glean additional insights. Various other image-based detections (e.g., object detection) can be performed to draw out differences in protein structures, for example, which can further be used to perform the modeling techniques described herein. Sometimes, the insights and additional information gleaned from processing the x-ray data can be combined with traditional tabular data to generate more accurate and/or robust modeling results/outcomes.

As another example, the computer system can leverage graph neural networks to process the x-ray data. The graph neural network includes a network structure that shows linkage between and amongst various data points in the x-ray data and in various dimensions (in comparison to tabular data, which can be two dimensional (2D) and contain columns and rows, thereby limiting how much data is collected and/or available for further analysis). The graph neural networks may leverage graphical information about proteins in the x-ray data, such as height and other dimensional data apparent in a graph. Therefore, the protein data need not be converted into 2D tabular, structural format and more data points can be processed and analyzed to attain modeling results/outcomes described herein. The computer system may receive or retrieve (e.g., from a data store) homological models with known amino acid (AA) sequences. As another example, the computer system may perform an Alphafold search, such as identifying a large number of protein structures in a similar family.

The computer system may then model protein properties based on the collected data about the protein structures, especially when the protein structures unfold when temperature increases (block 306). The properties may include pH and/or pI. Modeling the protein properties may include performing molecular dynamics simulations. The simulations may include coarse-grained structure creation, equilibrium of molecular systems such as number of moles (N), volume (V), and temperature (T) being kept constant in molecular dynamic simulations (NVT), or such as number of moles (N), pressure (P), and temperature (T) being kept constant in molecular dynamic simulations (NPT), and/or 500 ns, and/or replicas of different molecule systems. Any of these simulations can provide for protein titration based on pH setup. Such simulations can also calculate charge based on force fields and parameters. An equilibrium procedure can be performed as follows, in some implementations: dissipative particle dynamics (DPD) calculation, coarse-grained structures creation, and NPT at a range of temperature and run 500 ns.

Optionally, the computer system may perform one or more post-analysis techniques based on the protein properties (block 308). For example, the computer system may perform a function of g(r) by disulfide S—S bonds from cysteine. As another example, the computer system may perform aggregation by hydrophobic calculation. As other examples, the computer system may perform solubility calculations, determine clusters and numbers of molecules in clusters, and/or determine surface areas (hydrophobic and/or hydrophilic) of the proteins and/or their respective protein properties. Additionally or alternatively, the post-analysis techniques may be performed on other structural protein properties, such as protein disulfide bonding, hydrophobic surface interaction, aggregation, radial distribution function, number of reacted disulfide bonds, aggregation cluster size, and/or surface properties.

The computer system may retrieve at least one model in block 310. At least one model may include a temperature model, pH model, and/or protein-content model. At least one model may be any other type of model described herein, which may include a model that was trained according to various parameters, including but not limited to temperature, pH, and protein-content.

The computer system may apply the model(s) to the protein properties in block 312. In some implementations, the computer system may provide additional inputs to the model(s). For example, computer-analytic, sensory, and/or consumer data may be provided as inputs to generate more robust model outputs. Multi-dimensional predictions and/or feature importance may also be performed as part of applying the model(s) to the protein properties. The protein properties and other data may also be augmented using existing or historical data and/or chemistry-based models to expand and improve accuracy of model determinations and outputs. Similarly, the protein properties may be provided to the model(s) as input along with molecular data (e.g., graphs, images) associated with the protein structures.

The computer system may determine, based on applying the model(s), instructions for developing the food product (block 314). As described herein, the instructions may include recommended concentrations of the protein structures and/or of the plant ingredients for use in developing the food product. The instructions may include recommended processing conditions for developing the food product with the protein structures and/or with the plant ingredients (e.g., temperature, pressure, time).

In block 316, the computer system may return the instructions. Returning the instructions may include storing the instructions in a data store. Returning the instructions may include transmitting the instructions or other information from the process 200 to a user device for presentation in one or more GUIs.

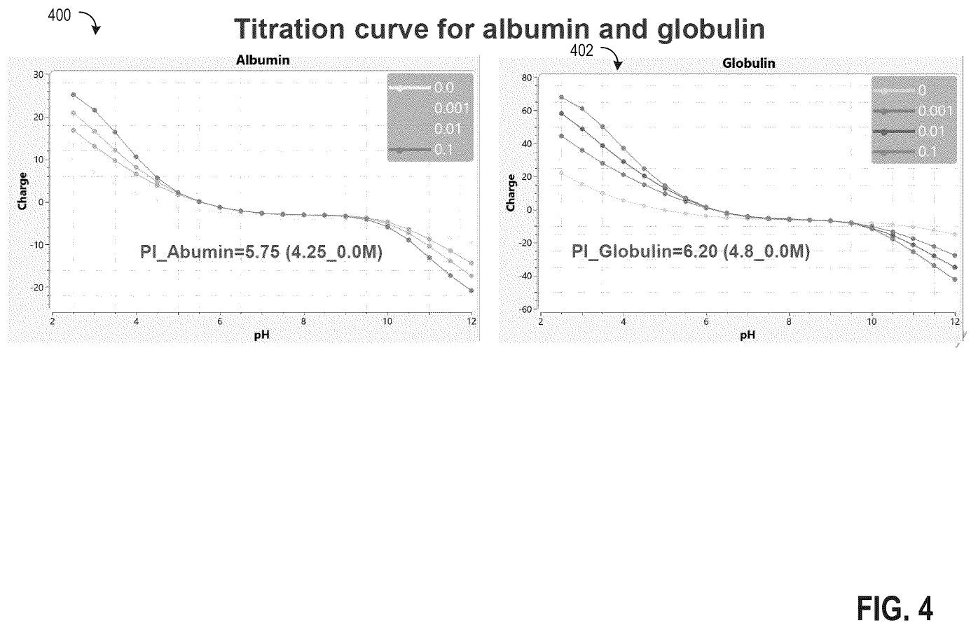

FIG. 4 Graphs show the impact of the pH and salt content on, for example, albumin and globulin using the disclosed techniques. More specifically, graph 400 shows the impact of the pH and salt content on albumin and graph 402 shows the impact of the pH and salt content on globulin.

Graphs 400 and 402 show results from modeling different pH levels of the albumin and globulin in chickpeas. The described modeling techniques may be used to predict the change of the solubility of the chickpea protein with respect to different pH levels and how that may affect how much of the chickpea protein is added to a product formula or product mixture for developing a particular food product, such as dough for chips or other snacks. The described modeling techniques may also be used to determine different concentrations of salt that may be added to the formula or mixture to reduce or increase, i.e., modulate the solubility of the plant ingredient proteins. In some implementations, different concentrations of acid may be added to the formula to adjust the pH to a level to achieve a desired solubility.

Accordingly, if pH is very low (e.g., less than a predetermined threshold pH level), the proteins (e.g., albumin, globulin) are more soluble, may have a charge of 0, and will repel rather than aggregate. Once a preferred, predetermined quantity of salt is added to the formula or mixture, the pH may increase. An increased pH level may cause the proteins to have a charge above 0, which may impact the degree and amount of aggregation and consequently a desired level of solubility. The disclosed models may be used to predict what pI should exist for each chickpea protein being added to the formula or mixture for developing the food product. The disclosed models may also be used to determine a desired pH of the food product, based on the pH, salt, and/or quantity of the chickpea protein(s) that is added to the formula or mixture.

In an illustrative example of the disclosed technology, a predicted pI may be determined by the following formula: pI=a1*5.75+a2*6.20, where a1 is a ratio of albumin in an entire protein and a2 is a ratio of globulin in an entire protein. Other types of proteins may also be modeled using the disclosed techniques. Salt concentration may not impact pI, as shown in both the graphs 400 and 402. However, no-salt/high salt (e.g., >0.1 M) may impact the pI of both albumin and globulin. A buffer range may exist of approximately pH 6.5-9 in which the charge may not change. Such findings may be used to determine that pH may be adjusted closer to PI to achieve a best aggregation (e.g., less soluble) of a plant ingredient, such as when cooking chickpea proteins for use in developing the food product.

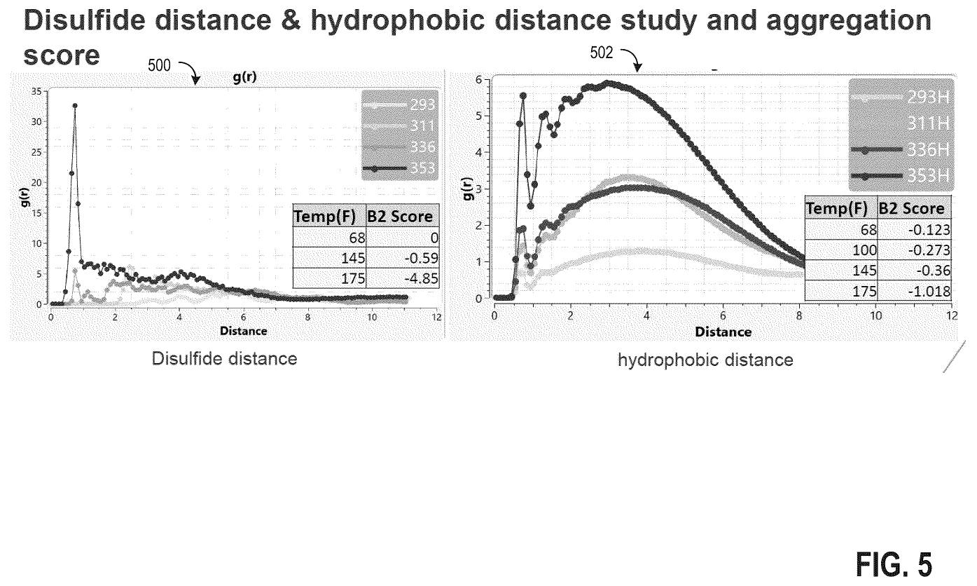

FIG. 5 illustrates graphs of the temperature impact on, for example, the disulfide and hydrophobic distances using the disclosed techniques. More specifically, graph 500 shows temperature impact on the disulfide distances for chickpea proteins and graph 502 shows temperature impact on the hydrophobic distances for chickpea proteins.

The graphs 500 and 502 show results from predicting solubility based on g(r) radial distribution function of disulfide bonding and protein surface properties. Temperature modeling may therefore be used to determine thermal impact on using plant ingredient proteins in development of a food product. Temperatures during processing of the plant ingredient proteins may be modeled and mapped to different levels of solubility, the levels of solubility impacting qualities/characteristics of the developed food product, such as texture, stickiness, mouthfeel, etc. At lower temperatures, such as a temperature of approximately 145° F., the resulting food product, such as dough, may be more sticky and more soluble. The high solubility of globulin and/or albumin can be due to hydrophilicity of surface amino acid residues and the relatively lower molecular weight, comparing to aggregated protein at higher temperatures. Pure proteins may not aggregate at lower temperatures (e.g., 145° F.), so there may be more hydrophilic areas to interact with water. At the higher temperatures, such as a temperature of 175° F., aggregation levels may be higher/stronger and cause less solubility.

In the illustrative example of FIG. 5, solubility decreased with increasing temperature values. High disulfide interaction (covalent bond), shown in the graph 500, indicates strong aggregation at a temperature of approximately 175° F. High hydrophobic interaction (surface interaction), shown in the graph 502, indicates relatively strong aggregation at the temperature 175° F. Higher temperature may result in more aggregation. Aggregation starts actively near the protein denaturation temperature (Td) and may be caused by a protein (e.g., albumin, globulin) unfolding, S—S bonding, and/or hydrophobic interaction.

FIG. 6 illustrates graphs of protein content impact; for example, disulfide and hydrophobic aggregation, interaction, and solubility. More specifically, graph 600 shows protein content impact on disulfide aggregation and solubility. Graph 602 shows protein content impact on hydrophobic interaction and solubility. Graphs 604 and 606 show protein content impact on disulfide aggregation and solubility. Graph 608 shows protein content impact on hydrophobic interaction and solubility. In the examples shown in the graphs 600 and 602, 50% of chickpea was used, resulting in 10% of protein content. In the examples shown in the graphs 604, 606, and 608, 100% of chickpea was used, resulting in 20% protein content.

Protein content may be modeled using the disclosed technology with different quantities/percentages of protein in the plant ingredient (e.g., chickpea) to determine a formula for developing the food product with the plant ingredient protein. As shown in the graphs 600, 602, 604, 606, and 608, solubility may increase when more chickpea is added to the formula, thereby causing the resulting food product (e.g., dough) to become more sticky. The amount of chickpea may be adjusted to predict and achieve a desired level of solubility.

Aggregation occurs strongly in low protein content. High protein content may cause limited aggregation. Solubility changing point (e.g., higher aggregation) may occur after Td in low protein content formulas. For the low protein content formulas, the processing time and temperature may be decreased to achieve the desired solubility for developing the food product with the plant ingredient protein (e.g., proteins in chickpeas).

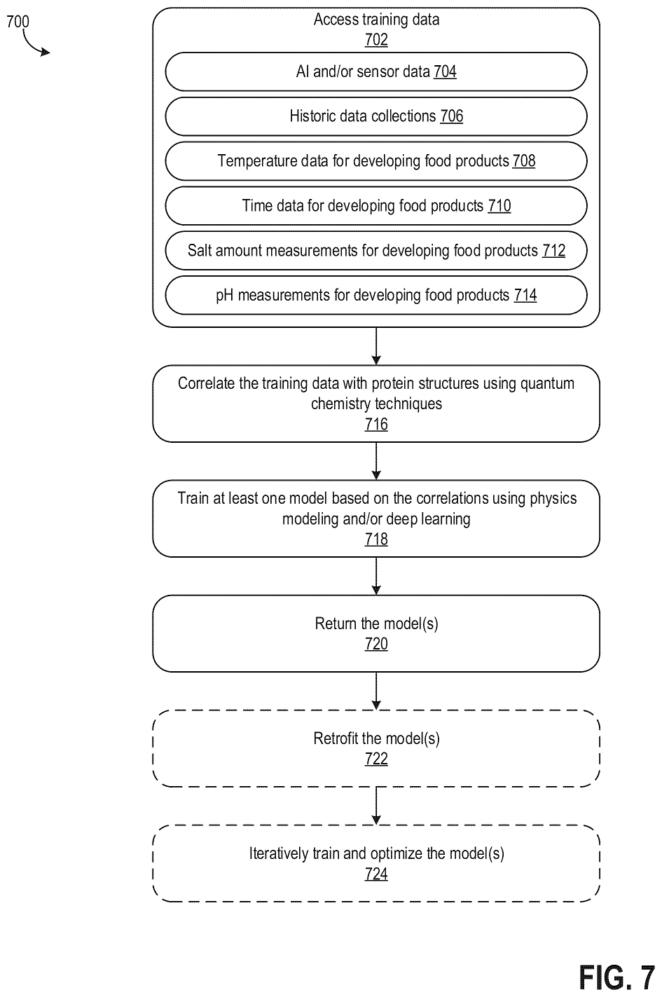

FIG. 7 is a flowchart of a process 700 for training at least one model to predict plant ingredient conditions for developing food products. The process 700 may be used to train any of the models described herein, such as a temperature model, a pH model, and/or a protein content model. In some implementations, the process 700 may be used to train a deep learning model or any other type of model that may predict any type of plant ingredient conditions for developing any type of food product based on any one or more of temperature, pH, and/or protein content. Sometimes, a model may be trained for predicting conditions of a specific type of plant ingredient (e.g., chickpeas, oats, legumes, lentils, peas, faba beans). Sometimes, a model may be trained for predicting conditions of a specific type of plant protein (e.g., albumin, globulin). Sometimes, a model may be trained for predicting plant ingredient conditions (e.g., temperature, pressure, etc.) for developing a particular type of food product.

The process 700 may be performed by the computer system 102. The process 700 may also be performed by one or more other computing systems, devices, computers, networks, cloud-based systems, and/or cloud-based services. For illustrative purposes, the process 700 is described from the perspective of a computer system.

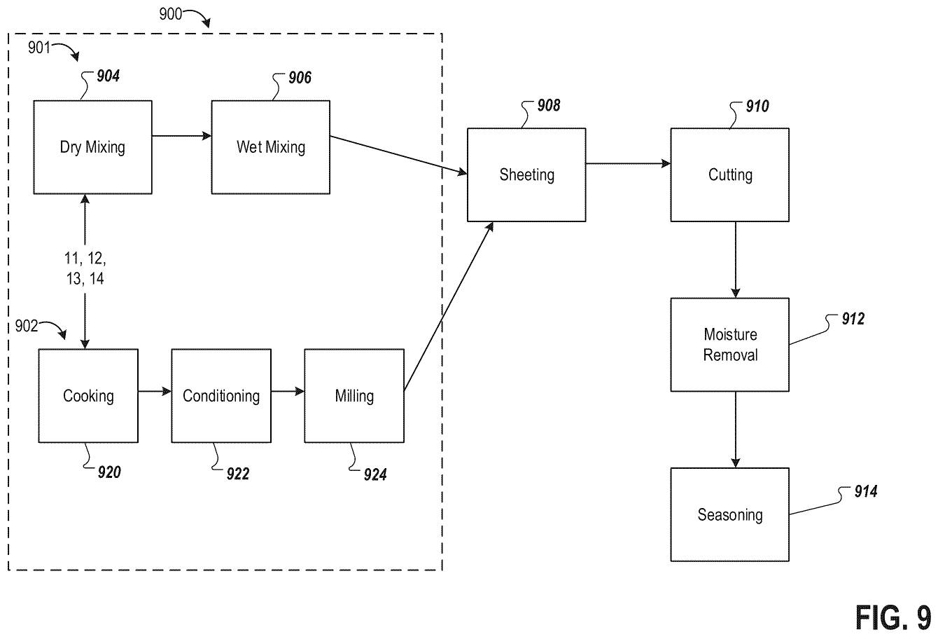

Referring to the process 700, the computer system may access training data in block 702. The training data may include, but is not limited to, AI and/or sensor data (block 704), historic data collections (block 706), temperature data for developing food products (block 708), time data for developing food products (block 710), salt amount measurements for developing food products (block 712), and/or pH measurements for developing food products (block 714). The AI and/or sensor data (block 704) may include temperature values that are measured by temperature sensors/probes in one or more unit operations that are used for developing a food product with the plant ingredient. Refer to FIG. 9 for further discussion about exemplary unit operations. Sometimes, the sensor data may include pressure values that are measured by pressure sensors in the one or more unit operations that are used for developing the food product with the plant ingredient. Various other sensor data collected by IoT devices can be used. Sometimes, one or more of the data accessed in block 702 can be augmented to generate synthetic data, which can be used to further enhance training of models described herein. The historic data collections (block 706) may include any measurements of temperature, salt, pH, protein content, PI, pressure, etc. that are gathered using sensing devices and/or calculations during prior developments of the food products with plant ingredients. Similarly, any of the temperature data (block 708), time data (block 710), salt amount measurements (block 712), and/or pH measurements (block 714) may be previously measured during development of the food products with the plant ingredients, stored in a data store described herein, and then retrieved in the process 700.

In block 702, the computer system may collect and aggregate a variety of data from a variety of different sources. One or more of the training data in blocks 704-714 may include data, including but not limited to public data about crystal structures and different temperature values of the plant ingredients used in developing the food products.

The computer system may correlate the training data with protein structures using suitable techniques, including but not limited to quantum chemistry techniques in block 716. The quantum chemistry techniques may include molecular dynamics simulation with molecular mechanisms. The training data may be correlated with the protein structures in the plant ingredients to determine how the proteins change during food product development (e.g., during food product processing including food product processing conditions) and how the protein structures are impacted by different reactions, interactions with other ingredients, processing conditions and the like. Sometimes, correlating the training data with the protein structures may include labeling and/or annotating the training data with various proteins structures and/or labeling/annotating the protein structures with the different reactions, processing conditions, etc. For example, albumin and globulin proteins may be labeled as less soluble at particular high temperatures (e.g., 175° F.) based on training data indicating that a desired level of solubility of the resulting developed food product (e.g., dough) was achieved at the particular high temperatures. Composition of a mixture and ratio of various ingredients may also impact dough surface properties.

In block 718, the computer system may train at least one model based on the correlations using physics modeling and/or other AI-related techniques. Where time-related data is used for training and/or run-time model use, the model(s) can be trained as a long-short term memory network/model (LSTM). Convolutional neural network (CNN) and/or deep neural network (DNN) models may also be trained for prediction capabilities. Shapely values may also be leveraged to interpret the model(s) and gain insights on feature importance, such as what parameters may drive and/or contribute more than others to model output (e.g., predictions).

The computer system may return the model in block 720. Returning the model may include storing the model in a data store described herein. Returning the model may include providing and using the model during runtime/in real-time.

Optionally, the computer system may retrofit the model(s) (block 722). The model(s) may be retrofitted as part of iteratively improving and/or training the model(s). The model(s) may be retrofitted for pure proteins. For various protein-containing formulas, more ingredients may be added to the described modeling to re-train or otherwise improve the model. Simulations using the model(s) can provide trends of impacts on protein content, water content, pH, temperature, and/or humidity as well as other known and used parameters.

Optionally, the computer system may iteratively train and optimize the model(s) (block 724). The model(s) may be iteratively trained in a feedback loop in which the model(s) is used during runtime. The feedback loop may also provide for monitoring model drifting (e.g., deviation from expected formulas, ingredients, or other model outputs). Iteratively training the model(s) may be performed using different concentrations of the plant ingredient. Iteratively training the model(s) may be performed using data resulting from developing the food product according to runtime instructions that are generated and/or recommended by the computer system as described herein. The data used for training and improving the model may include a breakdown of the formula (e.g., compound or molecular level), analytical data, processing conditions data, sensory data, and/or consumer data. Training and improving the model with this type of data may help achieve insights relating to consumer-centric and/or human-centric product innovation. The model(s) may also be retrofitted using one or more other iterative training and/or optimization processes.

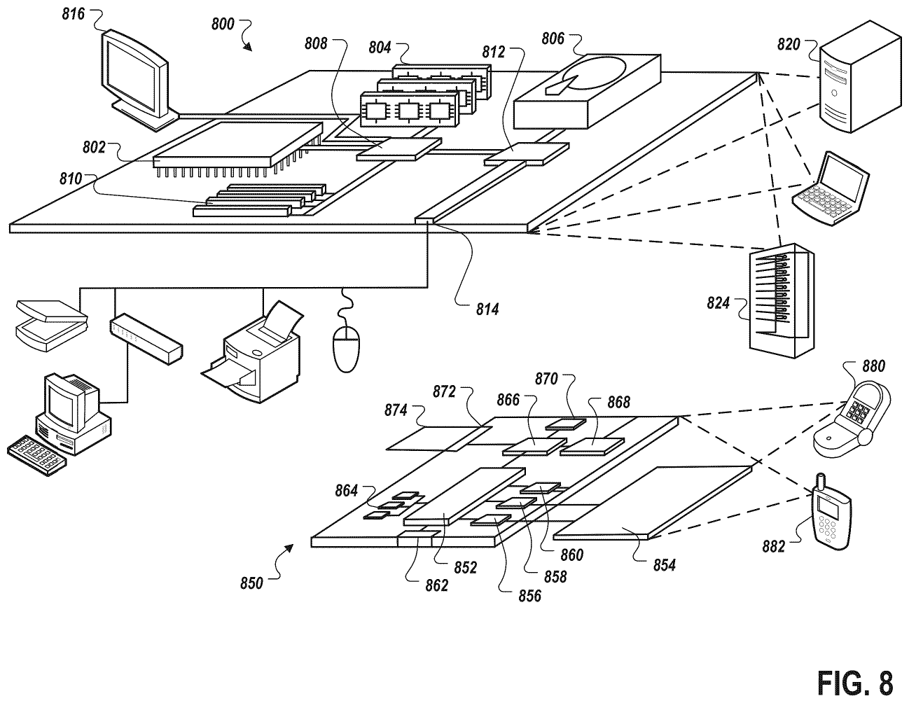

FIG. 8 shows an example of a computing device 800 and an example of a mobile computing device that may be used to implement the described techniques. The computing device 800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and may be mounted on a common motherboard or in other manners as appropriate. The processor 802 may process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 804 stores information within the computing device 800. In some implementations, the memory 804 is a volatile memory unit or units. In some implementations, the memory 804 is a non-volatile memory unit or units. The memory 804 may also be another form of computer-readable medium, such as a magnetic or optical disk.

The storage device 806 is capable of providing mass storage for the computing device 800. In some implementations, the storage device 806 may be or contain a computer-readable medium, such as a floppy disk device, a hard disk device, an optical disk device, or a tape device, a flash memory or other similar solid state memory device, or an array of devices, including devices in a storage area network or other configurations. A computer program product may be tangibly embodied in an information carrier. The computer program product may also contain instructions that, when executed, perform one or more methods, such as those described above. The computer program product may also be tangibly embodied in a computer- or machine-readable medium, such as the memory 804, the storage device 806, or memory on the processor 802.

The high-speed interface 808 manages bandwidth-intensive operations for the computing device 800, while the low-speed interface 812 manages lower bandwidth-intensive operations. Such allocation of functions is exemplary only. In some implementations, the high-speed interface 808 is coupled to the memory 804, the display 816 (e.g., through a graphics processor or accelerator), and to the high-speed expansion ports 810, which may accept various expansion cards (not shown). In the implementation, the low-speed interface 812 is coupled to the storage device 806 and the low-speed expansion port 814. The low-speed expansion port 814, which may include various communication ports (e.g., USB, Bluetooth, Ethernet, wireless Ethernet) may be coupled to one or more input/output devices, such as a keyboard, a pointing device, or a networking device such as a switch or router, e.g., through a network adapter.

The computing device 800 may be implemented in a number of different forms, as shown in the figure. For example, it may be implemented as a standard server 820, or multiple times in a group of such servers. In addition, it may be implemented in a personal computer such as a laptop computer 822. It may also be implemented as part of a rack server system 824. Alternatively, components from the computing device 800 may be combined with other components in a mobile device (not shown), such as a mobile computing device 850. Each of such devices may contain one or more of the computing device 800 and the mobile computing device 850, and an entire system may be made up of multiple computing devices communicating with each other.

The mobile computing device 850 includes a processor 852, a memory 864, an input/output device such as a display 854, a communication interface 866, and a transceiver 868, among other components. The mobile computing device 850 may also be provided with a storage device, such as a micro-drive or other device, to provide additional storage. Each of the processor 852, the memory 864, the display 854, the communication interface 866, and the transceiver 868, are interconnected using various buses, and several of the components may be mounted on a common motherboard or in other manners as appropriate.

The processor 852 may execute instructions within the mobile computing device 850, including instructions stored in the memory 864. The processor 852 may be implemented as a chipset of chips that include separate and multiple analog and digital processors. The processor 852 may provide, for example, for coordination of the other components of the mobile computing device 850, such as control of user interfaces, applications run by the mobile computing device 850, and wireless communication by the mobile computing device 850.

The processor 852 may communicate with a user through a control interface 858 and a display interface 856 coupled to the display 854. The display 854 may be, for example, a TFT (Thin-Film-Transistor Liquid Crystal Display) display or an OLED (Organic Light Emitting Diode) display, or other appropriate display technology. The display interface 856 may comprise appropriate circuitry for driving the display 854 to present graphical and other information to a user. The control interface 858 may receive commands from a user and convert them for submission to the processor 852. In addition, an external interface 862 may provide communication with the processor 852, so as to enable near area communication of the mobile computing device 850 with other devices. The external interface 862 may provide, for example, for wired communication in some implementations, or for wireless communication in other implementations, and multiple interfaces may also be used.

The memory 864 stores information within the mobile computing device 850. The memory 864 may be implemented as one or more of a computer-readable medium or media, a volatile memory unit or units, or a non-volatile memory unit or units. An expansion memory 874 may also be provided and connected to the mobile computing device 850 through an expansion interface 872, which may include, for example, a SIMM (Single In Line Memory Module) card interface. The expansion memory 874 may provide extra storage space for the mobile computing device 850, or may also store applications or other information for the mobile computing device 850. Specifically, the expansion memory 874 may include instructions to carry out or supplement the processes described above, and may include secure information also. Thus, for example, the expansion memory 874 may be provide as a security module for the mobile computing device 850, and may be programmed with instructions that permit secure use of the mobile computing device 850. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product may be a computer- or machine-readable medium, such as the memory 864, the expansion memory 874, or memory on the processor 852. In some implementations, the computer program product may be received in a propagated signal, for example, over the transceiver 868 or the external interface 862.

The mobile computing device 850 may communicate wirelessly through the communication interface 866, which may include digital signal processing circuitry where necessary. The communication interface 866 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 868 using a radio-frequency. In addition, short-range communication may occur, such as using a Bluetooth, WiFi, or other such transceiver (not shown). In addition, a GPS (Global Positioning System) receiver module 870 may provide additional navigation- and location-related wireless data to the mobile computing device 850, which may be used as appropriate by applications running on the mobile computing device 850.

The mobile computing device 850 may also communicate audibly using an audio codec 860, which may receive spoken information from a user and convert it to usable digital information. The audio codec 860 may likewise generate audible sound for a user, such as through a speaker, e.g., in a handset of the mobile computing device 850. Such sound may include sound from voice telephone calls, may include recorded sound (e.g., voice messages, music files, etc.) and may also include sound generated by applications operating on the mobile computing device 850.

The mobile computing device 850 may be implemented in a number of different forms, as shown in the figure. For example, it may be implemented as a cellular telephone 880. It may also be implemented as part of a smart-phone 882, personal digital assistant, or other similar mobile device.

Various implementations of the systems and techniques described here may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here may be implemented on a computer having a display device (e.g., a LED (light-emitting diode) display or LCD (liquid crystal display) monitor) for displaying information to the user and a keyboard and a pointing device (e.g., a mouse or a trackball) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

The systems and techniques described here may be implemented in a computing system that includes a back end component (e.g., as a data server), or that includes a middleware component (e.g., an application server), or that includes a front end component (e.g., a client computer having a graphical user interface or a Web browser through which a user may interact with an implementation of the systems and techniques described here), or any combination of such back end, middleware, or front end components. The components of the system may be interconnected by any form or medium of digital data communication (e.g., a communication network). Examples of communication networks include a local area network (LAN), a wide area network (WAN), and the Internet.

The computing system may include clients and servers. A client and server are generally remote from each other and typically interact through a communication network. The relationship of client and server arises by virtue of computer programs running on the respective computers and having a client-server relationship to each other.

FIG. 9 is a schematic diagram of exemplary methods for making a snack food according to one embodiment. The methods can be used to make baked snack foods and other snack foods, such as tortilla chips. In this regard, it will be appreciated that each block, unit of operation, and/or apparatus shown and described in FIG. 9 may not be required but is merely shown as an illustrative example of a process for developing or producing snack foods, such as chips, pretzels, etc., to which the described processes for determining a concentration of a plant ingredient in a food product before or while making the food product can be applied.

The method(s) illustrated in FIG. 9 includes a mixing operation 900 to form a dough that may then be sheeted 908. In one mixing embodiment 901, one or more ingredients 11, 12, 13, and 14, one of which may include the protein-containing plant ingredient, may be dry mixed 904 and thereafter combined with an aqueous solution in a wet mixer 906 to form a dough. In an illustrative example, one or more of the raw ingredients 11 12, 13, and 14 may include but is not limited to flours, sources of proteins, starches, and/or additives (e.g., leavening agents). Alternatively, the mixing of dry ingredients (904) and subsequent (or simultaneous) addition of water (906) to the mix of dry ingredients may occur in a same apparatus or unit of operation to form an uncooked snack food dough.

The uncooked dough may be sheeted during a sheeting operation 908, which can be performed by a sheeter. The resulting sheets can subsequently be cut into pieces by a cutter in a cutting operation 910. In some instances, the sheeting and cutting may occur simultaneously to provide an uncooked dough sheet with a plurality of cut pieces.

Thereafter, the sheeted uncooked snack food dough may undergo a moisture removal operation 912. The moisture content can be reduced to form a partially-cooked product using, for example, an oven. Sometimes, a fryer may additionally or alternatively be used to fry the partially-cooked product in oil. A seasoning operation 914 may also be performed before packaging.

It will be appreciated that one or more sensors may be associated with each, some, or all of the apparatuses and/or unit of operations used to perform the operations described in the method.

In an alternative mixing method 902, the raw ingredients 11, 12, 13, and/or 14, one of which may include the protein-containing plant ingredient may be provided to a unit of operation for cooking 920. The cooked ingredients may then be conditioned 922 (e.g., wash cooked seeds or other ingredients, de-germ, remove one or more elements from the ingredients) and then milled 924. The resulting dough can then be used to perform the operations 908, 910, 912, and 914 described above.

In some illustrative examples, an extruder may be used in developing the food products described herein. Ingredients may be worked through an application of mechanical and/or thermal energy in an extruder, after which the extrudate may be formed and cut into pieces by a cutter. Extrusion techniques may be used to generate some doughs, such as dough for pretzels, where very low pressure ranges (e.g., below a predetermined threshold level or range) may be used.

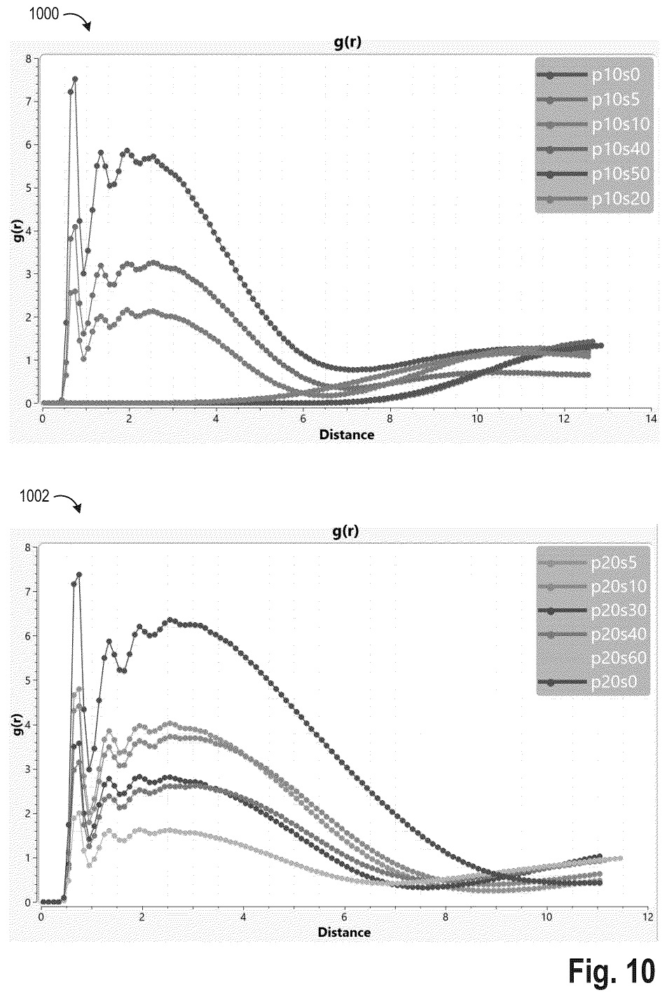

FIG. 10 illustrates graphs depicting results from modeling dough mixtures with various ratios of protein to starch. Sometimes, the process described in reference to FIG. 9 can use subsequent ovens or drying operations, which can further be subjected to moisture removal (operation 912) and to provide shelf stable products. Accordingly, the disclosed techniques can be used to model protein-starch interactions by hydrophobic surface, as shown in graphs 1000 and 1002. In the graph 1000, the protein is at an exemplary 10%, and the starch may be from 0 to 5, 10, 20, 30, 40, and/or 50%. Sometimes, when the starch content is greater than 10%, there may be no protein aggregation. In the graph 1002, the protein content is at an exemplary 20%, and the starch may be from 0 to 5, 10, 20, 30, 40, 50, and/or 60%. Protein aggregation may decrease with increased starch content, as shown by the results in the graph 1002. However, aggregation levels may not reach zero.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments may also be implemented in combination in a single embodiment in part or in whole. Conversely, various features that are described in the context of a single embodiment may also be implemented in multiple embodiments separately or in any suitable sub-combination. Moreover, although features may be described herein as acting in certain combinations and/or initially claimed as such, one or more features from a claimed combination may in some cases be excised from the combination, and the claimed combination may be directed to a sub-combination or variation of a sub-combination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

1. A method for determining a concentration of chickpeas in a developed food product before or while making the food product, the method comprising:
receiving protein data for the chickpeas;
retrieving, from a data store, at least one model that has been trained to generate output indicating a desired concentration of the chickpeas to be used in developing the food product, wherein the at least one model includes at least one of a pH model, a temperature model, and a protein-content model;
providing the received protein data as input to the at least one model;
receiving the output generated by the at least one model, wherein the output indicates the desired concentration range of the chickpeas to be used in developing the food product;
generating instructions for developing the food product, wherein the instructions include the desired concentration range of the chickpeas; and
returning the instructions.

2. The method of claim 1, wherein providing the received protein data to the at least one model comprises providing at least a portion of the received protein data to each of the pH model, the temperature model, and the protein-content model.

3. The method of claim 2, the method further comprising:
comparing output from each of the pH model, the temperature model, and the protein-content model; and
generating, based on the comparison, an updated desired concentration range of the chickpeas.

4. The method of claim 1, wherein the instructions further include at least one of (i) an amount of salt based on the desired concentration range of the chickpeas and (ii) a temperature above or below a threshold temperature value at which to develop the food product with the desired concentration range of the chickpeas.

5. The method of claim 1, wherein the pH model was trained to correlate a measured pH level during one or more unit operations performed to produce the developed food product with predetermined concentrations of salt that either reduce or increase solubility of the protein in the chickpeas to determine desired concentrations of the chickpeas to be included in a formulation to produce different food products.

6. The method of claim 1, wherein the at least one model is a deep learning model that was trained to determine a desired concentration for a plurality of different plant ingredients to be used in developing the food product, wherein the plurality of different plant ingredients comprises the chickpeas.

7. The method of claim 6, wherein the plurality of different plant ingredients include one or more proteins selected from the group consisting of albumin, globulin, prolamin, glutelin, and mixtures thereof.

8. The method of claim 1, further comprising:
receiving at least one processing condition for which the food product is developed and a desired amount of protein present in the developed food product; and
providing the received at least one processing condition and the desired amount of protein present as input to the at least one model.

9. The method of claim 8, wherein the at least one processing condition comprises a temperature in one or more unit operations used for developing the food product.

10. The method of claim 8, wherein the at least one processing condition comprises a pressure level in one or more unit operations used for developing the food product.

11. The method of claim 1, wherein the pH model was trained to: (i) predict different pH levels to be used when producing the developed food product based on using different concentrations of the chickpeas, (ii) determine a desired concentration range of the chickpeas to be used in developing the food product based on the predicted different pH levels of the ingredients used to form a dough used for developing the food product, and (iii) generate output indicating the determined desired concentration range of the chickpeas.

12. The method of claim 1, wherein the temperature model was trained to: (i) correlate (a) temperatures at which the food product is developed with different concentrations of the chickpeas with (b) solubility of the protein in the chickpeas to generate a temperature correlation, (ii) determine a desired concentration range of the chickpeas to be used in developing the food product based on the temperature correlation, and (iii) generate output indicating the determined desired concentration range of the chickpeas.

13. The method of claim 1, wherein the protein-content model was trained to: (i) correlate (a) proteins and amounts present in the chickpeas in developing the food product with (b) solubility of the proteins to generate a protein-content correlation, (ii) determine a desired concentration range of the chickpeas to be used in developing the food product based on the protein-content correlation, and (iii) generate output indicating the determined desired concentration range of the chickpeas.

14. The method of claim 1, wherein, returning the instructions comprises transmitting the instructions to a user device that is configured to present at least the desired concentration range of the chickpeas in a graphical user interface (GUI) display of the user device.

15. The method of claim 1, further comprising iteratively training the at least one model using (i) different concentrations of the chickpeas and (ii) data resulting from developing the food product according to the instructions.

16. A method for determining a concentration of a plant ingredient in a developed food product before or while making the food product, the method comprising:
receiving protein data for the plant ingredient;
retrieving, from a data store, at least one model that has been trained to generate output indicating a desired concentration of the plant ingredient to be used in developing the food product;
providing the received protein data as input to the at least one model;
receiving the output generated by the at least one model, wherein the output indicates the desired concentration range of the plant ingredient to be used in developing the food product;
generating instructions for developing the food product, wherein the instructions include the desired concentration range of the plant ingredient; and
returning the instructions.

17. The method of claim 16, wherein the at least one model comprises at least one of a pH model, a temperature model, and a protein-content model.

18. The method of claim 16, wherein the at least one model was trained using a deep neural network (DNN) to determine desired concentrations of different plant ingredients used for developing different food products, wherein the different plant ingredients include at least chickpeas.

19. The method of claim 16, wherein the instructions include an amount of salt based on the desired concentration range of the plant ingredient.

20. The method of claim 16, wherein the instructions include a temperature above or below a threshold temperature value at which to develop the food product with the desired concentration range of the plant ingredient.
